(19) United States
(12) Patent Application Publication
(10) Pub. No.: US 2009/0325211 A1
(43) Pub. Date: Dec. 31, 2009

(54) System and method for dual-detection of a cellular response

(76) Inventors: Ye Fang, Painted Post, NY (US); Ann M. Ferrie, Painted Post, NY (US); Joydeep Lahiri, Painted Post, NY (US); Deepti J. Mudaliar, Horseheads, NY (US); Qi Wu, Painted Post, NY (US)

Correspondence Address: CORNING INCORPORATED, SP-TI-3-1, CORNING, NY, 14831, US

(21) Appl. No.: 12/151,179

(22) Filed: May 5, 2008

Related U.S. Application Data
(60) Provisional application No. 60/997,974, filed on Oct. 6, 2007.
(60) Provisional application No. 61/010,442, filed on Jan. 9, 2008.

(51) Int. Cl. C12Q 1/02; C12M 1/34

(52) U.S. Cl. 435/29; 435/288.7

(57) ABSTRACT

A system and method as defined herein for dual-detection of evanescent-wave label-free light and evanescent-wave excited-fluorescent label-emitted light in an optical biosensor.

### CLAIMING BENEFIT OF PRIOR FILED U.S. APPLICATION

This application claims the benefit of U.S. Provisional Application Ser. No. 60/997,974, filed Oct. 6, 2007, and U.S. Provisional Application Ser. No. 61/010,442, filed Jan. 9, 2008. The content of this prior filed U.S. application and the entire disclosure of any publications, patents, and patent documents mentioned herein are incorporated by reference.

### BACKGROUND

The disclosure relates to optical biosensors, specifically resonant waveguide grating (RWG) biosensors, for detection of stimulus-induced responses of live-cells.

### SUMMARY

The disclosure provides a dual- or multi-modal system and method that can detect, for example, both evanescent wave (EW)-excited fluorescence and evanescent wave-based dynamic mass redistribution (DMR) signals of live-cells in response to, for example, stimulation. In embodiments, the disclosure provides methods that enable the study of cell-signaling, compound screening, and like processes, for a selected target and optionally in a high throughput format. The system and method provide signaling specificity and high information content. In embodiments, the disclosure provides a system and methods for dual- or multi-modal ion channel biosensor cellular assays. The disclosure also enables the detection of cellular responses using evanescent-wave excited-fluorescence with long excitation wavelengths, for example, greater than about 650 nm. The disclosed system and method are useful in a variety of applications including, for example, drug discovery, therapeutic efficacy evaluation such as ADME-Tox (absorption, distribution, metabolism, and excretion, and toxicity) studies, diagnostics, environmental trace analysis, bioterrorism detection, basic and applied research, and like areas.

### BRIEF DESCRIPTION OF THE DRAWINGS

FIG. 1 shows a schematic of a resonant waveguide grating (RWG) biosensor for simultaneously detecting both evanescent wave-excited fluorescence and evanescent-wave optical signals (i.e., DMR signal) as a result of dynamic relocation of cellular matter within the sensor volume in an immobilized live-cell, in embodiments of the disclosure.

FIGS. 2A to 2C show schematics of biosensor systems that enable both label-independent and label-dependent optical signals of live-cells in response to stimulation, in embodiments of the disclosure.

FIGS. 3A and 3B show the correlation between the resonant wavelength and the incident angle using the transverse magnetic (TM) mode, in embodiments of the disclosure.

FIGS. 4A and 4B show the correlation between the resonant wavelength and the incident angle using the transverse electric (TE) mode, in embodiments of the disclosure.

FIG. 5 shows the grating reflectivity spectra of transverse magnetic (TM) modes as the incident angle increases, from right to left, from 1 to 57 degrees in 1 degree increments, in embodiments of the disclosure.

FIGS. 6A and 6B show that shorter wavelength light can be resonantly coupled into a grating through second order diffraction, in embodiments of the disclosure.

FIGS. 7A and 7B respectively show a schematic of a RWG biosensor in a microplate array format, and a fluorescent image of a biosensor well having two discrete regions (reference and sample) using forward propagating TM mode with a resonant wavelength of 785 nm, in embodiments of the disclosure.

FIG. 8 shows the fluorescent intensity distribution across a scanned row of the sensor in FIG. 7B, in embodiments of the disclosure.

FIGS. 9A and 9B respectively show a fluorescent image of a biosensor well having two discrete regions using the forward propagating TE (transverse electric) mode with a resonant wavelength of 785 nm, and the scanned fluorescent intensity across the biosensor at a selected pixel range of FIG. 9A, in embodiments of the disclosure.

FIGS. 10A and 10B respectively show a fluorescent image of a biosensor well having two discrete regions using the forward propagating TM mode with a resonant wavelength of 790 nm, and the scanned fluorescent intensity distribution across the sensor at a selected pixel range, in embodiments of the disclosure.

FIGS. 11A and 11B respectively show a fluorescent image of a biosensor well having two discrete regions using the forward propagating TM mode with a resonant wavelength of 790 nm, and the fluorescent intensity distribution across the sensor at a selected pixel range, in embodiments of the disclosure.

FIGS. 12A to 12D show comparative fluorescence intensities of A431 cells cultured on biosensor microplate surfaces in response to stimulation with IRDye® labeled EGF over time, in embodiments of the disclosure.

FIGS. 13A to 13C shows schematics of a biosensor system that excites a membrane potential-sensitive dye in the visible region at the basal cell membrane surface, in embodiments of the disclosure.

FIGS. 14A to 14C shows schematics of a biosensor system that excites two fluorescence dyes in the visible region where one dye is membrane potential-sensitive and at the basal cell membrane surface, in embodiments of the disclosure.

FIGS. 15A to 15C shows schematics of a biosensor system that excites a membrane potential-sensitive dye in the visible region in the presence of a fluorescence quencher at the basal cell membrane surface, in embodiments of the disclosure.

### DETAILED DESCRIPTION

Various embodiments of the disclosure will be described in detail with reference to drawings, if any. Reference to various embodiments does not limit the scope of the invention, which is limited only by the scope of the claims attached hereto. Additionally, any examples set forth in this specification are not limiting and merely set forth some of the many possible embodiments for the claimed invention.

### DEFINITIONS

“Assay,” “assaying,” or like terms refer to an analysis to determine, for example, the presence, absence, quantity, extent, kinetics, dynamics, type, or like measures of a cell's label-dependent and label-independent response upon contact or stimulation with a stimulus, for example, an exogenous or endogenous stimuli, such as an antibody, an antibody mimic, a ligand candidate compound, a viral particle, a pathogen, or like entity.

“Attach,” “attachment,” “adhere,” “adhered,” “adherent,” “immobilized,” or like terms generally refer to immobilizing or fixing, for example, a surface modifier substance, a compatibilizer, a cell, a ligand candidate compound, or like entities of the disclosure, to a surface, such as by physical absorption, chemical bonding, and like processes, or combinations thereof. Particularly, “cell attachment,” “cell adhesion,” or like terms refer to the interacting or binding of cells to a surface, such as by culturing, or interacting with cell anchoring materials (e.g., extracellular matrices, adhesion complexes, etc.), a compatibilizer (e.g., fibronectin, collagen, lamin, gelatin, polylysine, etc.), and like materials, or a combination thereof.

“Adherent cells” refers to a cell, a cell line, or a cell system, such as a prokaryotic or eukaryotic cell, that remain associated with, immobilized on, or in certain contact with the outer surface of a substrate. Such type of cells after culturing can withstand or survive washing and medium exchanging process, a process that is prerequisite to many cell-based assays. “Weakly adherent cells” refers to a cell or a cell line or a cell system, such as a prokaryotic or eukaryotic cell, which weakly interacts, or associates with or contacts the surface of a substrate during cell culture. However, these types of cells, for example, human embryonic kidney (HEK) cells, tend to dissociate easily from the surface of a substrate by physically disturbing approaches such as washing or medium exchange. “Suspension cells” refers to a cell or a cell line that is preferably cultured in a medium wherein the cells do not attach or adhere to the surface of a substrate during the culture. “Cell culture” or “cell culturing” refers to the process by which either prokaryotic or eukaryotic cells are grown under controlled conditions. “Cell culture” not only refers to the culturing of cells derived from multicellular eukaryotes, especially animal cells, but also to the culturing of, for example, complex tissues and organs.

“Cell” or like term refers to a small usually microscopic mass of protoplasm bounded externally by a semipermeable membrane, optionally including one or more nuclei and various other organelles, capable alone or interacting with other like masses of performing all the fundamental functions of life, and forming the smallest structural unit of living matter capable of functioning independently including synthetic cell constructs, cell model systems, and like artificial cellular systems.

“Cell system” or like term refers to a collection of more than one type of cell or differentiated forms of a single type of cell, which interact with each other, thus performing a biological or physiological or pathophysiological function. Such cell system includes, for example, an organ, a tissue, a stem cell, a differentiated hepatocyte cell, or like systems, and a combination thereof.

“Antibody,” “Ab,” or like terms refer generally to a protein biomolecule, or a biomolecule mimic, typically having a Y-shaped and found in blood or other bodily fluids of vertebrates, including soluble, membrane bound, membrane-liberated, or like forms, and monoclonal, polyclonal, natural, synthetic, engineered, and like forms. Antibodies are used by the immune system to identify and neutralize foreign objects or pathogens, such as bacteria and viruses, by reaction with surface antigens.

“Marker” or like term refers to a molecule, a biomolecule, or a biological material that is able to modulate the activities of at least one cellular target (e.g., a G_{q}-coupled receptor, a Gₛ-coupled receptor, a Gᵢ-coupled receptor, a G_{12/13}-coupled receptor, an ion channel, a receptor tyrosine kinase, a transporter, a sodium-proton exchanger, a nuclear receptor, a cellular kinase, a cellular protein, etc.), and can result in a reliably detectable output or signal measurable by a biosensor. Depending on the class of the intended cellular target and its subsequent cellular event(s), a marker can be, for example, an activator, such as an agonist, a partial agonist, an inverse agonist, for example, for a G protein-coupled receptor (GPCR), a receptor tyrosine kinase (RTK), an ion channel, a nuclear receptor, a cellular enzyme adenylate cyclase, and like cellular targets. The marker can be, for example, a ligand that binds to and activates a specific target, or a molecule that binds to and activates another distinct target, which in turn transactivates the specific target.

“Detect,” “detection,” “detecting,” or like terms refer to an ability of the system, apparatus, and methods of the disclosure to discover or sense the interaction of a stimulus on a cellular target with a biosensor.

“Stimulus,” “therapeutic candidate compound,” “therapeutic candidate,” “prophylactic candidate,” “prophylactic agent,” “ligand candidate,” or like terms refer to a molecule or material, naturally occurring or synthetic, that is of interest for its potential to interact with a cell attached to the biosensor. A stimulus can also include an additional or alternative chemical agent, photochemical agent, mechanical agent, electrical agent, or a combination thereof. A therapeutic or prophylactic candidate can include, for example, a chemical compound, a biological molecule, a peptide, a protein, a biological sample, a drug candidate small molecule, a drug candidate biologic molecule, a drug candidate small molecule-biologic conjugate, and like material or molecular entity, or combinations thereof, which can specifically bind to or interact with at least one of a cellular target or a pathogen target such as a protein, DNA, RNA, an ion, a lipid, or like structure or component of a live-cell.

“Biosensor” or like terms refer to a sensor device for the detection of an analyte or interaction that can combine a biological component with a physicochemical detector component. The biosensor can be typically comprised of three parts: a biological component or element (such as tissue, microorganism, pathogen, cells, or combinations thereof); a detector element (which operates, e.g., in a physicochemical manner such as optical, piezoelectric, electrochemical, thermometric, magnetic, etc.); and a transducer associated with both components. The biological component or element can include, for example, a live-cell, a pathogen, or a combination thereof. In embodiments, an optical biosensor can comprise an optical transducer for converting a molecular recognition or molecular stimulation event in, for example, a live-cell, a pathogen, or a combination thereof, into a detectable and quantifiable signal.

“Epidermal growth factor” or “EGF” refers to a growth factor that plays a significant role in the regulation of cell growth, proliferation, and differentiation. Human EGF is a 6,045 Da protein having 53 amino acid residues and three intramolecular disulfide bonds. EGF acts by binding with high affinity to EGFR on the cell surface and stimulating the intrinsic protein-tyrosine kinase activity of the receptor. The tyrosine kinase activity in turn initiates a signal transduction cascade which results in a variety of biochemical changes within the cell, such as a rise in intracellular calcium levels, increased glycolysis and protein synthesis, and increases in the expression of certain genes, including the gene for EGFR that can ultimately leads to DNA synthesis and cell proliferation.

“Epidermal growth factor receptor” or “EGFR” refers to a particular receptor on the cell's surface that can be activated by binding of its specific ligands, including EGF and transforming growth factor α (TGFα). The EGF receptor is a member of the ErbB family of receptors, a subfamily of four closely related receptor tyrosine kinases: EGFR (ErbB-1), HER2/c-neu (ErbB-2), Her 3 (ErbB-3) and Her 4 (ErbB-4). The related ErbB-3 and ErbB-4 receptors are activated by neuregulins (NRGs). ErbB-2 has no known direct activating ligand, and may be in an activated state constitutively. Upon activation by its growth factor ligands, EGFR undergoes a transition from an inactive monomeric form to an active homodimer, although there is evidence that preformed inactive dimers may also exist before ligand binding. In addition to forming homodimers after ligand binding, EGFR may pair with another member of the ErbB receptor family, such as ErbB2/Her2/neu, to create an activated heterodimer. There is also evidence to suggest that clusters of activated EGFRs form, although it remains unclear whether this clustering is important for activation itself or occurs subsequent to activation of individual dimers.

“Transactivation” or like terms refer to the activation of a receptor (e.g., EGFR) triggered by a ligand that binds to and activates another distinct cell receptor (e.g., a GPCR). As a result of cellular regulatory machineries, the former receptor becomes transactivated. Such transactivation is a common principle in communication between different cellular signaling systems that enables cells to integrate a multitude of signals from its environment. For example, transactivation of the EGFR represents the paradigm for cross-talk between GPCRs and RTKs (see for example, Gschwind, A., et al., “Cell Communication Networks: Epidermal Growth Factor Receptor Transactivation as the Paradigm for Interreceptor Signal Transmission,” Oncogene, (2001), 20 (13), 1594-1600). Another example is the transactivation of Kv 1.2 potassium ion channel in HEK 293 cells with carbachol, a GPCR muscrunic receptor ligand. A transactivating ligand, transactivating marker, or transactivating molecule refer to a ligand, marker, or molecule that can activate a target receptor of interest indirectly, possibly through intracellular regulatory or signaling mechanism(s), rather than directly binding to and activating the target receptor.

Abbreviations, which are well known to one of ordinary skill in the art, may be used (e.g., “h” or “hr” for hour or hours, “g” or “gm” for gram(s), “mL” for milliliters, and “rt” for room temperature, “nm” for nanometers, and like abbreviations).

The indefinite article “a” or “an” and its corresponding definite article “the” as used herein means at least one, or one or more, unless specified otherwise.

“Include,” “includes,” or like terms means including but not limited to.

“About” modifying, for example, the quantity of an ingredient in a composition, concentrations, volumes, process temperature, process time, yields, flow rates, pressures, and like values, and ranges thereof, employed in describing the embodiments of the disclosure, refers to variation in the numerical quantity that can occur, for example: through typical measuring and handling procedures used for making compounds, compositions, concentrates or use formulations; through inadvertent error in these procedures; through differences in the manufacture, source, or purity of starting materials or ingredients used to carry out the methods; and like considerations. The term “about” also encompasses amounts that differ due to aging of a composition or formulation with a particular initial concentration or mixture, and amounts that differ due to mixing or processing a composition or formulation with a particular initial concentration or mixture. Whether modified by the term “about” the claims appended hereto include equivalents to these quantities.

“Consisting essentially of” in embodiments refers to, for example, a system for evanescent-wave label-free light and evanescent-wave excited-fluorescence light detection as defined herein; an apparatus for characterizing a live-cell including the aforementioned system as defined herein; a method for characterizing a live-cell as defined herein; a method to enhance detection of a single resonant wavelength of an EW-label-free signal and an EW-excited fluorescence signal from a single sensor; and articles, devices, or apparatus of the disclosure, and can include the components or steps listed in the claim, plus other components or steps that do not materially affect the basic and novel properties of the compositions, articles, apparatus, and methods of making and use of the disclosure, such as particular reactants, particular additives or ingredients, a particular agent, a particular cell or cell line, a particular surface modifier or condition, a particular ligand candidate, or like structure, material, or process variable selected.

Thus, the claimed invention may suitably comprise, consist of, or consist essentially of, any of:

a system for evanescent-wave label-free light and evanescent-wave excited-fluorescence light detection as defined herein;

an apparatus for characterizing a live-cell including the aforementioned system as defined herein;

a method for characterizing a live-cell as defined herein;

a method to enhance detection of a single resonant wavelength of an EW-label-free signal and an EW-excited fluorescence signal from a single sensor; and

a method for detection of ion-channel activity in a live-cell.

This application is related in certain aspects to the following commonly owned and assigned patent applications:

U.S. patent application Ser. No. 11/027,547, filed Dec. 29, 2004, entitled “Spatially Scanned Optical Reader System and Method for Using Same,” Publication No. US 20060141611 A1, published Jun. 29, 2006.

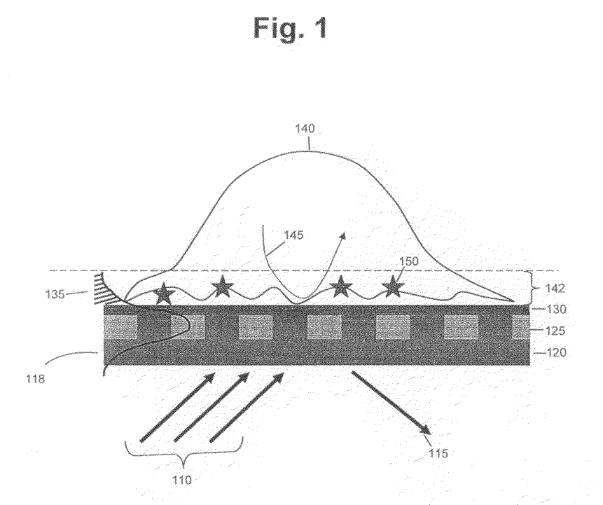

U.S. patent application Ser. No. 11/027,509, filed Dec. 29, 2004, entitled “Method for Creating a Reference Region and a Sample Region on a Biosensor and the Resulting Biosensor”, Publication No. US 20040141527 A1, published Jun. 29, 2006, see for example FIG. 1 which illustrates three different methods for creating a reference region and a sample region on a single biosensor.

U.S. patent application Ser. No. 11/210,920, filed Aug. 23, 2005, entitled “Optical Reader System and Method for Monitoring and Correcting Lateral and Angular Misalignments of Label Independent Biosensors”, Publication No. US 20060139641 A1, published Jun. 29, 2006, mentions an optical reader system that uses a scanned optical beam to interrogate a biosensor to determine if a biomolecular binding event occurred on a surface of the biosensor. In embodiments, the optical reader system can include, for example, a light source, a detector, and a processor (e.g., computer, digital signal processor (DSP)). The light source outputs an optical beam which is scanned across a moving biosensor while the detector collects the optical beam which has been resonantly reflected from the biosensor. Alternatively, the light source outputs an optical beam which illuminates a whole sensor while the detector images the optical beams across the whole sensor which have been resonantly reflected from the biosensor. The processor processes the collected optical beam and records the resulting raw spectral or angle data which is a function of a position (and possibly time) on the biosensor. The processor can then analyze the raw data to create a spatial map of resonant wavelength (peak position) or resonant angle which indicates whether or not a biomolecular binding event or a cellular event occurred on the biosensor. Several other uses of the raw data are also described.

U.S. Patent Application Ser. No. 60/781,397, filed Mar. 10, 2006, entitled “Optimized Method for LID Biosensor Resonance Detection,” now U.S. patent application Ser. No. 11/716,425, filed Mar. 9, 2007.

U.S. Patent Application Ser. No. 60/844,736, filed Sep. 9, 2006, entitled “Active Microplate Position Correction for Biosensors.”

U.S. patent application Ser. No. 11/711,207, filed Feb. 27, 2007, entitled “Swept Wavelength Imaging Optical Interrogation System and Method for Using Same.”

Specific and preferred values disclosed for components, ingredients, additives, cell types, antibodies, and like aspects, and ranges thereof, are for illustration only; they do not exclude other defined values or other values within defined ranges. The compositions, apparatus, and methods of the disclosure include those having any value or any combination of the values, specific values, more specific values, and preferred values described herein.

The disclosure provides a biosensor system that simultaneously detects both stimulus-induced optical signals independent of labels, and fluorescent signals dependent on labels in immobilized or living cells. Both the label-dependent and the label-independent detection use an evanescent wave arising from a biosensor. The disclosure provides methods that are suitable for high throughput system (HTS) analysis of cellular responses, and for screening drugs or compounds that typically can interfere with an optical signal, with a fluorescent signal, or both. The disclosure also describes methods that enable a wide spectrum of evanescent-wave excited-fluorescence applications.

The disclosure provides methods for label-free and non-invasive optical biosensor-based cell assays having high signal specificity. These biosensors are label-free, and can provide an integrated cellular response, referred to as dynamic mass redistribution (DMR) for probing cell biology. The DMR signal can consist of many contributions from cellular processes downstream of the activation of a receptor. Hence, these biosensor-based cell assays can be considered to be “non-specific” relative to a single cellular process which is typically measured using conventional cell assays. By measuring evanescent-wave excited-fluorescence associated with a specific cellular process, biosensor-based cell assays can be integrated with traditional cell assays. The combined measurements offer complimentary and corroborative information, and provide new insights into cell biology discussed below.

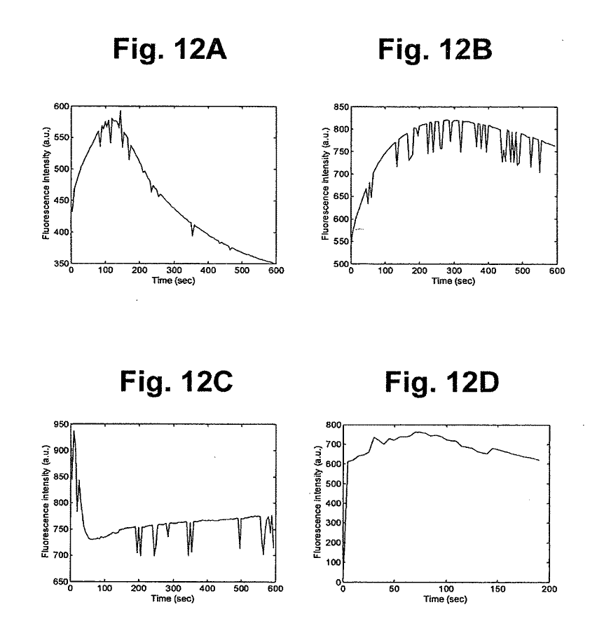

Cell signaling was originally thought to function via linear routes where a single extracellular signal would trigger a linear chain of reactions resulting in a single well-defined response. However, on-going research has shown that cellular responses to external stimuli are considerably more complicated, and are the result of multiple interacting pathways containing many common molecules. These pathways do not simply transmit, but can for example, process, encode, and integrate internal and external signals. Cells rely on highly dynamic networked interactions in their response to stimulation from external signals. The combinatorial integration of signaling pathways mediated through a specific molecule in response to stimuli plays an important role in the specificity of cellular responses and cell functions, for example, the signaling of epidermal growth factor receptor (EGFR). Upon ligand binding, the EGFR can become dimerized and activated through auto-phosphorylation of the receptor on tyrosine residues in the cytoplasmic domain, thus initiating a number of intracellular signals by interacting with distinct signaling proteins. However, the specificity of cell responses is largely determined by the integration of signaling network interactions, and depends on the cellular context. As expected, RWG biosensor label-independent assays show that under a quiescent state, obtained using 0.1% fetal bovine serum, stimulation of A431 cells with EGF led to a dose-dependent DMR signal that exhibits slower kinetics and smaller amplitudes (Fang, Y., et al., Anal. Chem. 2005, 77, 5720-5725), compared to those obtained in fully quiescent A431 cells using 0% fetal bovine serum (Fang, Y., Biophys. J, 2006, 91, 1925-1940). In contrast, chemical-biology studies that use chemical compounds to selectively modulate the activity of intracellular targets in the EGFR signaling pathways provide a link between the EGF-induced DMR signal to specific signaling pathways downstream EGFR. The EGF-induced DMR signal requires EGFR tyrosine kinase activity, actin polymerization, and dynamin, and mainly proceeds through MEK. Furthermore, the positive-DMR phase (P-DMR; an increased signal over time) is primarily due to the translocation of intracellular targets to the activated receptors, while the negative-DMR phase (N-DMR; a decreased signal over time) is due to the combination of receptor internalization and cell detachment. These chemical-biology analyses indicate that the EGF-induced DMR signal is not related to a single and specific cell signaling event; rather, it represents the combinatorial integration of many cellular events downstream of the EGFR activation. As a result, the kinetic parameters of each of the DMR phases are difficult to link to a specific cellular event; and the overall DMR signal can be viewed as non-specifically related to a signaling pathway or a single signaling event, but it is specific to the EGFR target (Fang, Y., et al., Anal. Chem., 2005, 77, 5720-5725). Conversely, the evanescent-wave excited-fluorescence measurements, as shown in the present disclosure using NIR-dye labeled EGF (FIG. 12), indicate that the change in fluorescence intensity over time after addition of NIR-dye labeled EGF is primarily associated with two major events. Although not limited by theory the initial increase in fluorescence intensity is believed to be due to the binding of fluorescently labeled EGF to the receptors located at the basal cell membrane of cultured cells, and the subsequent decrease in fluorescence intensity is believed to be due to the internalization of receptors together with the bound fluorescent EGF. These results indicate that the EGF molecules can diffuse and bind to the EGFR located at the basal membrane surface within the sensor detection zone (the top membrane surface is far away from the sensor surface, and thus the binding of dye labeled EGF cannot be easily detected using current sensor configurations), thus leading to the increase in fluorescence intensity, whose kinetics is similar to those for the P-DMR phase using the label-independent DMR measurement. Moreover, the time for internalization to occur is consistent with the findings for the transition time from the P-DMR to the N-DMR event for fully quiescent cells (0% fetal bovine serum). The receptor internalization is believed to take the bound labeled EGF inside the cells, thus leading to a decrease in fluorescence intensity. These results show that both label-independent and label-dependent assays can confirm each other, and can also provide specificity to the distinct cellular events of downstream EGFR signaling to label-independent measurements. Furthermore, both measurements in combination offer an integrated picture how EGF binds to the receptors at cell surface, and how and when EGFR signaling proceeds.

In embodiments, the disclosure provides a dual-detection system for evanescent-wave label-free light and evanescent-wave excited-fluorescence light detection, the system comprising:

an optical sensor;

a light source to illuminate the sensor;

an optical detection system or detector to collect the evanescent-wave label-free light and evanescent-wave excited-fluorescence light from the sensor; and

a processor to analyze the collected light.

In alternative embodiments, the disclosure provides a dual-detection system for evanescent-wave label-free light and evanescent-wave excited-fluorescence light detection, the system comprising:

an optical sensor;

a light source to illuminate the sensor;

an optical detection system or detector to collect the evanescent-wave label-free light from the sensor;

a second detection system or detector to collect evanescent-wave excited-fluorescence light from the sensor; and

a processor to analyze the collected light.

The optical sensor can be, for example, a single optical sensor, an array of waveguide grating coupled sensors on a microplate, and like configurations. The light source can be, for example, a fiber coupled tunable laser system, such as multiple tunable lasers or a combination of a plurality of tunable lasers having, for example, wavelengths from about 400 to about 900 nanometers, and like wavelengths, or wavelength segments or ranges therein. The light source's illumination of the sensor is selected such that it excites a fluorescent label having direct or indirect association with the surface of the sensor and provides evanescent-wave label-free light associated with a dynamic mass redistribution event (DMR) of a cell associated with the sensor surface. The optical detection system or detector can include, for example, a self-referenced interferometer. The self-referenced (i.e., wavelength referencing) interferometer can be used to dynamically measure laser light source wavelengths, see for example, U.S. Pat. No. 5,305,074. The light collected by the optical detection system can be, for example, evanescent-wave label-free light, evanescent-wave fluorescence label emitted-light, or a combination thereof.

Typical components of a fluorescence detection and imaging system can include, for example, a light source (e.g., xenon or mercury arc-discharge lamp), an excitation filter, a dichroic mirror (or dichromatic beam splitter), and an emission filter. The filters and the dichroic can be selected to match the spectral excitation and emission characteristics of the fluorophore used to label the stimulus. In embodiments, the dual-detection system can include, for example, a first beam splitter that adjusts the incident angle of the light source's excitation beam and a second beam splitter that selects EW-label-free reflected light and EW-excited fluorescence label emitted light.

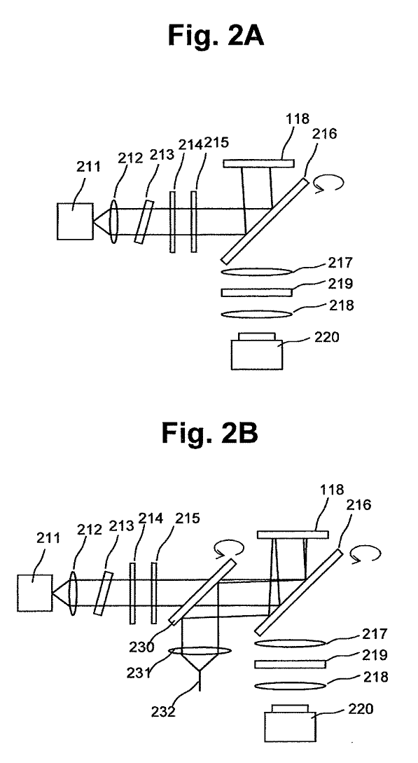

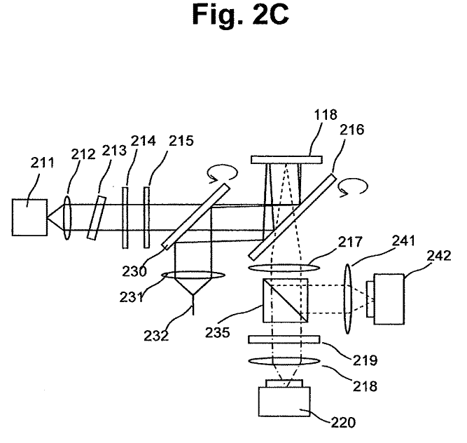

In embodiments of the dual-detection system, the detection system or detector for collecting EW-label-free light can be separate from, or integral with, the optical detection system or detector for collecting EW-excited fluorescence label light. In embodiments the optical detection system or detector can include, for example, a first digital camera for collecting EW-label-free light and a second digital camera for collecting EW-excited fluorescence label light, reference for example, FIG. 2C.

An optical sensor can comprise one or more sensors, including an array of sensors, for example, as used in a microplate article having a plurality of attached or embedded biosensors, or like articles or applications.

A suitable light source can be, for example, a tunable light source, such as a tunable laser adapted to illuminate one or more of the sensors in a swept wavelength fashion, such that each biosensor within an array of biosensors can be systematically illuminated, for example, simultaneously, in a regular sequence, or in groups. Although the resonant wavelengths may differ from sensor to sensor within an array, the laser beam can be passed through optional illumination optics as disclosed herein so that the laser beam can be expanded to illuminate a substantial part of the sensor area or the entire sensor area. Thus, the tunable illumination source includes illumination optics.

An example of an optical detection system for collection of light from the sensor can be, for example, a digital camera having an area scan image sensor. The digital camera, having an area scan image sensor with digitized outputs, can record, for example, the spectral images as the tunable laser scans the sensor. In embodiments the digital camera can include imaging optics which conditions the resulting sensor light image for receipt and recordation by the digital camera.

In embodiments, the system can optionally further include at least one of or a combination of: a collimating lens; an excitation filter having a bandwidth of ±1 nm; an optical shutter; a polarization controller; a imaging lense; a notch filter; and a fluorescence emission filter.

In embodiments the disclosure provides an apparatus for characterizing a live-cell, the apparatus comprising:

the above mentioned system and any or all of the optional components, the system having a live-cell associated with a surface of the sensor, such as the side of the surface opposite the side of the surface being illuminated.

In embodiments the disclosure provides a method for characterizing a live-cell, the method comprising:

providing a system in accordance with the above mentioned system having a live-cell immobilized on the sensor's surface;

providing a first fluorescent-labeled stimulus for a selected target associated with the live-cell immobilized on the sensor's surface;

contacting the immobilized cell with the first fluorescent-labeled stimulus;

detecting the effect of the first fluorescent-labeled stimulus contact on the target by interrogating the sensor for EW-label-free light and EW-fluorescence light; and

comparing the sensor's EW-label-free light and EW-excited fluorescence light in the presence and absence of a second stimulus.

In embodiments, the second stimulus can be used as an additional probe, such as a labeled- or unlabeled-stimulus, but at least a different stimulus from the first fluorescent-labeled stimulus. Both the first and second stimuli can be added, for example, separately or simultaneously. When there are two addition steps, the order of stimulus addition can be dependent on the application. In embodiments the labeled stimulus can be, for example, added first for assays designed to determine the effect of a labeled stimulus on the second stimulus. Conversely, the second stimulus can be introduced first, for assays designed to determine the impact of the second stimulus on the fluorescent-labeled stimulus-induced optical output signals.

In embodiments the disclosure provides a method for characterizing a live-cell, the method comprising:

providing a “live-expression” system in accordance with the above mentioned system having a live-cell immobilized on the sensor's surface, where the live-cell expresses, such as actively, intermittently, or previously, a fluorescent target;

contacting the immobilized cell with a stimulus;

detecting the effect of the stimulus on the fluorescent target by interrogating the sensor for EW-fluorescence light; and

detecting the stimulus induced changes in the EW-label-free light.

In embodiments the stimulus can bind to and activate a different cellular target, i.e., other than the fluorescent target. The activation of the stimulus-binding target can trigger the translocation of the fluorescent target towards the basal cell membrane surface, or away the basal cell membrane surface, or out of the sensing volume of the biosensor, depending for example on the cellular localization of the fluorescent target. The expression of the fluorescent target in the live-cell can be achieved, for example, using gene expression vectors containing, for example, a fluorescent protein, such as green fluorescent protein (GFP), yellow fluorescent protein (YFP), red fluorescent protein (RFP), a long wavelength fluorescent protein (e.g., near IR fluorescent protein), or like fluorescent protein, or using a transfection approach to directly deliver fluorescent probes or fluorescent proteins into the cell, or using an incorporation approach to selectively incorporate fluorescent lipid molecules into the cell surface membrane. The incorporation approach can take advantage of fluorescent lipid molecules, such as membrane potential sensitive dye molecules or fluorescent tagged lipid molecules (e.g., Cy5-labeled 1,2-dipalmitoyl phosphatidylethanolamine (Cy5-DMPE), coumarin-linked phospholipids (CC2-DMPE)) or nanogold tagged lipids, which can directly insert into the cell surface membrane due to the strong lipid-lipid interactions.

In embodiments the disclosure provides a method to enhance detection of a single resonant wavelength of an EW-label-free signal and an EW-excited fluorescence signal from a single sensor, the method comprising:

measuring the EW-excited fluorescence signal of a specific target having a fluorescence label, and measuring the label-free dynamic mass redistribution signal upon stimulation; and

correlating the measured fluorescence signal arising from the target and the label-free optical signal (DMR signal).

In embodiments, the disclosure provides a method to enhance detection of a single resonant wavelength of an evanescent-wave label-free signal and an evanescent-wave excited-fluorescence signal from a single sensor, the method comprising:

measuring the evanescent-wave excited-fluorescence signal of a specific target having a fluorescent label, and measuring the label-free dynamic mass redistribution signal upon stimulation; and

correlating the fluorescence signal and the label-free dynamic mass redistribution signal.

Correlating the fluorescence signal and the label-free dynamic mass redistribution signal can include, for example, at least one of: comparing the kinetic profiles of both signals; comparing the modulation profiles of both signals by alteration of signaling cascades; comparing the impact of a gene alteration on the cellular response; or a combination thereof.

In embodiments the disclosure provides a method for characterizing a live-cell, the method comprising:

providing the above mentioned biosensor system having a live-cell immobilized on the biosensor's surface, the live-cell having a fluorescent target;

contacting the immobilized cell with a stimulus;

detecting the stimulus induced changes on the fluorescent target by interrogating the sensor for evanescent-wave fluorescence light; and

detecting the stimulus induced changes in the evanescent-wave label-free light. In embodiments, the live-cell having a fluorescent target can be accomplished, for example, with a gene expression vector which expresses a fluorescent protein. In embodiments, the live-cell having a fluorescent target can be accomplished, for example, with transfection methods to deliver a target into the live-cell, with insertion of a lipid target into the cell surface membrane, or a combination thereof.

In embodiments, the disclosure provides a dual-detection system for evanescent-wave label-free light and evanescent-wave excited-fluorescence light detection, the system comprising:

an optical sensor;

a light source to illuminate the sensor;

a first optical detector to collect the evanescent-wave label-free light from the sensor;

a second detector to collect evanescent-wave excited-fluorescence light from the sensor; and

a processor to analyze the collected light.

The optical sensor can include, for example, a patterned reference region, a sample region having a live-cell or a biomolecule thereon, or a combination thereof.

In embodiments the disclosure provides a system for dual-detection of ion-channel activity in a live-cell as disclosed herein.

In embodiments, the disclosure provides a method for dual-detection of ion-channel activity in a live-cell, the method including, for example:

providing a biosensor having at least one live-cell immobilized on the biosensor surface;

furnishing, such as imbibing, the immobilized cell with a membrane-potential sensitive dye;

contacting the immobilized cell having the imbibed membrane-potential sensitive dye with a stimulus; and

detecting the stimulus-induced optical label-free signal and evanescent wave excited fluorescence signal.

In embodiments, the disclosure provides a method for dual-detection of ion-channel activity in a live-cell, the method comprising:

providing a biosensor having at least one live-cell immobilized on the biosensor surface;

imbibing or otherwise furnishing the immobilized live-cell with a membrane-potential sensitive dye and a fluorescent lipid;

contacting the imbibed immobilized cell with a stimulus; and

detecting the stimulus-induced optical label-free signal and evanescent wave excited fluorescence signal, the fluorescence signal changes in relation to a change in fluorescent resonant energy transfer between the dye and the lipid.

In embodiments, the disclosure provides a method dual-detection of ion channel activity in a live-cell, the method comprising:

providing a biosensor having at least one live-cell immobilized on the biosensor surface;

imbibing or otherwise furnishing the immobilized cell with a membrane-potential sensitive dye and a quencher lipid;

contacting the imbibed immobilized cell with a stimulus; and

detecting the stimulus-induced optical label-free signal and evanescent wave excited fluorescence signal, the detected fluorescence signal changes in relation to a change in distance between the quencher and the membrane-potential sensitive dye.

In embodiments the disclosure provides a RWG biosensor having a visible light source having a nominally normal incident angle on the biosensor for detection of both evanescent-wave optical DMR and evanescent-wave excited fluorescence signals. The fluorescence signal can be generated from a membrane-potential sensitive dye having at least one visible excitation wavelength. The membrane potential sensitive dye can be, for example, within the basal cell membrane. In embodiments, a pair of fluorescent lipids, such as a membrane potential sensitive dye and a fluorescent lipid, are capable of fluorescence resonance energy transfer when in close proximity can be used to further enhance assay latitude or detection sensitivity. In embodiments, a pair of membrane incorporating molecules, such as a membrane-potential sensitive dye and a fluorescence quencher lipid can be selected. The quencher can quench the fluorescence of the membrane potential sensitive dye when in close proximity and can be used, for example, to further enhance assay latitude or detection sensitivity.

In embodiments the disclosure provides a system and method for dual-detection or multi-modal detection of ion channel activity in a live-cell using an evanescent-wave biosensor. The disclosure provides a system and method having increased assay sensitivity over conventional fluorescence ion channel cell assays due to the localized excitation of fluorescence molecules associated with a basal cell membrane surface. In addition, because of the ability to simultaneously detect ion channel DMR and the membrane potential-mediated fluorescence signals, the disclosed methods may reduce, for example, false positives and false negatives typically encountered using conventional fluorescence cell assay methodologies.

In embodiments the disclosure provides a system and method that are capable of detecting both evanescent wave-excited fluorescence and evanescent wave-based dynamic mass redistribution (DMR) signals of living cells that are specifically and directly linked to ion channel activity. The system and method use specific a RWG biosensor designed for visible wavelength light incident on the biosensor at nominally normal angle, in combination with the use of a single membrane potential-sensitive dye, or a pair of donor and acceptor dyes that is capable of fluorescence resonance energy transfer when they are located in proximity. The system and method enable the detection of both a label-free optical response and a membrane potential-associated fluorescence signal induced by, for example, an ion-channel opener, such as a ligand, an electric potential, a mechanical force, and like instrumentality, or a combination thereof.

Ion channels present a group of targets having major clinical indications, but which have been difficult to address due to a lack of suitable rapid but biologically significant assay methodologies. Ion channels regulate the movement of ions across biological membranes and play a crucial role in maintaining and modulating cellular function. Despite the significance of ion channels as drug targets, high-throughput screening for ion channel modulators has proven difficult and time-consuming, especially for voltage-gated ion channels. Heretofore, patch clamping methods have been a mainstay method. Such electrophysiological techniques remain restricted to screening a relatively low number of samples per day. Radioligand binding assays are disadvantaged by a requirement of prior knowledge of binding sites, and because other sites can be allosterically coupled. Thus, potentially valuable leads can be missed.

Functional assays are ideally suited to discover modulators of ion channel function, but available radioactive efflux assays require high amounts of radiotracer to load the cells. The conversion of the ⁸⁶Rubidium-efflux assay, routinely applied to study potassium channels to a non-radioactive format using atomic absorption spectroscopy, has removed the need for radioactive tracers but the method remains limited in throughput. One approach to measure ion channel function is the use of microphysiometry, in which the change of the extracellular pH in response to any change in the cells can be monitored. Although readers are commercially available and the assay is applicable to a broad range of targets, the throughput remains limited due to the length of measurements and the large number of cells required. The advance of fluorescent assays has significantly enhanced the portfolio of suitable assay technologies.

Activity of calcium channels can be measured using calcium-sensitive dyes, for example, Fluo-3 on systems such as the Fluorescent Imaging Plate Reader (FLIPR). Indirect measurement of membrane potential can be achieved with the FLIPR using, for example, oxonol dyes from the DiBac series (bis-barbituric acid oxonols), which show a change in distribution with changes in membrane potential that can be followed by a whole-cell image. Using a voltage-sensitive fluorescent probe(s) the activity of a channel can be monitored using Aurora's Voltage/Ion Probe Reader (VIPR). However, since these fluorescence measurements are carried out at the whole cell level, these systems require the application of voltage-sensitive fluorescence dyes coupled with fluorescence resonance energy transfer to achieve robust assays.

### System and Method

In embodiments the disclosure provides a system and method for measurement of both evanescent-wave optical radiation, such as refractive index changes, and evanescent-wave fluorescence radiation based on interrogation or imaging of a biosensor surface region, including emission detection and analysis. The interrogation of the surface region can be achieved by, for example, two distinct and complementary methods. In embodiments the interrogation can be accomplished by scanning the biosensor surface to construct an image of the sensor surface. In embodiments the interrogation can be accomplished by simultaneously obtaining an image of the refractive index changes from the biosensor surface and the fluorescence emission from the biosensor surface. The system and method of the disclosure can be use to perform, for example, diagnostic or therapeutic assays, such as for scanning evanescent-wave label-independent detection and scanning evanescent-wave fluorescence detection. In embodiments one or more biosensors can be situated in a well of a microplate and the disclosed system and method can be used to interrogate one or more of the biosensors to provide binding information between a target present on or in close proximity to the biosensor surface and a prospective binding analyte. In embodiments the disclosed system and method can be used to provide interaction or signaling information between a live-cell attached to the biosensor surface and a stimulus.

In embodiments the disclosure provides a method for analyzing a biosensor, for example, to determine the presence and extent of redistribution of cellular material within a live-cell, the method comprising:

irradiating and scanning the biosensor having a live-cell associated, such as immobilized, with the surface of the biosensor;

simultaneously detecting the evanescent-wave label-free signal and detecting the evanescent-wave fluorescence signal; and

correlating the label-free signal and the fluorescence signal with redistribution of cellular material.

In embodiments correlating signals with cellular redistribution can be accomplished by a correlation analysis that can be achieved by several different approaches, for example: comparing the kinetic parameters of both signals, since both signals represent a change of cellular response over time; comparing the modulation profiles of the cellular response by a modulator, such as an inhibitor or activator for a cellular target in the signaling pathways mediated through the target with which the stimulus interacts; comparing the impact of an alteration of a gene on the cellular response, such as a gene silencing using gene-knockout or interference-RNA, or a gene over-expression using gene transfection approaches, where for example, the gene encodes a cellular protein that is in a signaling cascade mediated through the stimulus-interacting target, and like approaches, or a combination thereof.

The label-free optical (light) signal represents an integrated cellular response that consists of many downstream signaling events, particularly those having significant relocation of cellular material (i.e., mass), mediated through the stimulus-induced activation of a specific cellular target, such as a GPCR or a receptor tyrosine kinase. In contrast, the fluorescence signal is directly associated with a specific cellular process such as binding of the fluorescent molecule to its target, the relocation of the fluorescent molecule in response to stimulation, or both events. However, cell signaling can be encoded by a series of spatial and temporal events, and the cellular regulatory machineries can play essential roles in integrating cellular responses. Therefore, both signals may share common kinetic profiles. For example, the transition time from an initial P-DMR event to the subsequent N-DMR event in the EGF-induced optical signal, as measured using RWG biosensor in quiescent A431 cells, was found to be associated with the receptor desensitization process, i.e., a process that is regulated by the phosphorylation of signaling cascades. Such phosphorylation is also required for receptor internalization process. As shown in FIG. 12A, the evanescent wave-excited fluorescence measurement for the labeled EGF-induced response also gives rise to an almost identical transition time from the initial increase in fluorescence intensity to the subsequent decrease in fluorescence intensity, in which the later change is due to receptor internalization. Thus, a kinetic analysis can correlate the EW-enabled light signal with the EW-excited fluorescent signal. Furthermore, the pretreatment of A431 cells with a dynamin inhibitor (dynamin inhibitory peptide) significantly attenuates both the decrease phases in both P-DMR and N-DMR signals (see Fang, Y. et al. Anal. Chem., 2005, 77, 5720-5725; and FIG. 12B). Such modulation profile by the dynamin inhibitor provides evidence that correlates the N-DMR event in the EGF (labeled or unlabeled)-induced EW-enabled light signal with the decrease in fluorescence of the labeled EGF-induced EW-excited fluorescence signal.

In embodiments, the biosensor contact surface can be, for example, undeveloped or unmodified. Thus, the method of the disclosure can provide a useful tool for determining, for example, baseline or reference data relating to the quality of a biosensor in neat, undeveloped, or unused microplates, or like manufactured surfaces. Additionally or alternatively, the biosensor contact surface can be developed or modified, for example, in advance, in situ, or both. Thus, for example, the method of the disclosure can be a useful tool for determining, for example, the quality of the data obtained from a biosensor in a microplate in, for example, a chemical, pharmalogical, biological, or like assay. Biosensor surface patterning can be generated by selectively blocking a specific area of the biosensor with a chemical or material that prevents the immobilization of a target of interest, and thus the binding of analytes to the immobilized target. Additionally or alternatively, patterning can be used to prevent the attachment of a live-cell, and thus the provision of a response of the live-cell to a stimulus. For example, a biosensor can be coated with a polymer such as EMA (poly(ethylene-alt-maleic anhydride)) reactive towards primary amines and a small area of the whole biosensor is then blocked with a small molecule, such as aminoethanol, through a conventional contact printing or stamping approach. In another example, the biosensor can be coated with a polymer such as SMA (styrene-maleic anhydride copolymer) that is reactive towards primary amines and a small portion of the biosensor surface is then blocked with a polyethylene glycol having an amine terminus using, for example, a contact printing or stamping approach, followed by the coating of the remainder of the surface with an extracellular matrix (ECM) material such as fibronectin, collagen, or gelatin. The resulting pre-blocked area becomes resistant to cell-adhesion and the cultured cells selectively bind to the ECM material presenting area.

In embodiments the biosensor can comprise, for example, a plurality of biosensors within a microplate, such as having 96- or 384-wells, or similar count wells including single wells, multi-wells, or compound wells. Additionally or alternatively, the biosensor can comprise any other suitable format.

In embodiments, the disclosure provides a system to determine live-cell responses, the system comprising:

a microplate comprising a frame including a plurality of wells formed therein, each well incorporating a biosensor having a surface with a reference region and a sample region;

a live-cell culture on the biosensor having one or more cells attached only to the sample region;

an optical reader-interrogator comprising an optical beam and optics for illuminating a portion of the biosensor, image optics for receiving reflected resonant light and EW excited-fluorescent light from the illuminated biosensor, and an imaging device for scanning and capturing a sequence of images from the illuminated biosensor; and

a processor to process the acquired scanned data in accordance with any of the methods of the disclosure.

In embodiments, the disclosure provides an optical interrogation system for a sensor, such as a biosensor, the system comprising:

an illuminator that emits an optical beam towards the biosensor;

a receiver that collects, separately or collectively, an optical beam and EW excited-fluorescent light from the biosensor and then outputs a signal which corresponds to the collected optical beam and fluorescent light; and

a processor to process the signal to determine live-cell responses in accordance with any of the methods of the disclosure.

In embodiments, the processor can be, for example, a programmable computer, a digital signal processor (DSP), or like devices for calculating, computing, comparing, selecting, or like operations of the system and the method.

In embodiments of the disclosure, a Corning Epic® label-independent detection system can be used as a label-independent biochemical binding detection system. It can consist of a 384-well microplate with optical biosensors within each well, and an optical reader to interrogate these microplates. Each well can contain a small (e.g., about 2 mm×2 mm) optical grating, known as a resonant waveguide grating (RWG). The wavelength of the light reflected by the grating is a sensitive function of the optical refractive index at the surface of the sensor inside the well. Hence, when a material such as a protein, antibody, drug, cell, or like material binds to the well bottom or sensor surface, the resonantly reflected wavelength will change. Alternatively, when a stimulus such as a compound, a drug, a biological, a drug candidate, a therapeutic agent, or like stimulus reacts with or interacts with the live-cell or a target associated with a live-cell attached to the biosensor surface, the resonantly reflected wavelength may change.

An optical reader, referred to as SLID for scanned label-independent detection, can use one or more focused optical beams that are scanned across the bottom (i.e., the opposite side from the immobilized cell or sample) of the microplate to measure reflected wavelength from each optical sensor. The reader may be used to monitor changes in the reflected wavelength from each sensor as a function of time. It may also be used to evaluate wavelength or changes in wavelength as a function of position within each sensor, that is, spatially resolved or imaging information.

When a biochemical material binds to the surface of a sensor the local refractive index is altered, and the wavelength reflected by the optical sensor changes. The reader detects and quantifies this wavelength change in order to measure biochemical events within each well. Light that impinges upon the sensor is resonantly coupled into the waveguide if it has the appropriate combination of wavelength and incident angle (i.e., wave vector.)

By monitoring the reflected wavelength (or angle) as a function of time, one may determine if material has bound to or has been removed from the surface of the sensor. A typical assay can be performed by first immobilizing, for example, a protein or a cell on the biosensor surface of microplate. Then a baseline read or measurement is accomplished where the wavelength reflected by each of the sensors in the microplate is measured and recorded. Then a binding compound (e.g., a drug compound or candidate) or stimulus is added to the wells, and a second wavelength read is accomplished. The wavelength shift that occurs between the two reads is a measure of how much drug or stimulus has bound to the surface of the biosensor of the microplate. Similarly but fundamentally different, in a cell-signaling study, a live-cell is brought into contact with the sensor surface. After culture, a baseline read is accomplished where the wavelength reflected by each biosensor in the plate is measured and recorded. Then a stimulus is introduced to the wells having the live-cells, and a second wavelength read can be accomplished either continuously (kinetic measurement) or discontinuously (such as an end-point measurement). The wavelength shift or wavelength difference before and after stimulation is a measure of the response of the live-cells attached on the sensor surface.

In embodiments, if desired, a portion of each sensor can be chemically or physically blocked to prevent binding, for example, of a target of interest or attachment of a live-cell. The blocked area can act as a reference signal for removing false wavelength shifts that can arise from environmental changes such as bulk refractive index changes, material drift, non-specific compound binding, thermal events, or like events. The interrogation system must be able to distinguish the signals from the sample and reference regions, each of which may occur at almost any wavelength within the sensor bandwidth, and can be of the same polarization. In embodiments, intra-well references, where a small portion of each well can be, for example, chemically blocked, can act as a spatially local reference.

Over several decades various label-free optical biosensors have been developed that provide detailed information of, for example, the binding affinity and kinetics of biomolecular interactions. These biosensors are often referred as affinity-based biosensors. Continuing improvements in biosensor instrumentation and experimental design have allowed a wider variety of interactions to be analyzed in greater detail. One example is the ability to directly detect the binding of small molecules to immobilized receptors and is therefore particularly useful in drug screening.

As drug discovery paradigms have begun to shift from a target-directed approach to a systems-biology centered approach, optical biosensors have seen increased uses for cell-based assays (see e.g., Fang, Y., (2006) Assays and Drug Development Technologies, 4: 583-595). The ability of label-free optical biosensors to examine stimulus-induced responses of live-cells is based upon the sensitivity of the biosensor's evanescent wave to detect changes in local mass density or distribution of the live-cell within its sensing volume or penetration depth. Resonant waveguide grating (RWG) biosensors have been applied to, for example, the study of activation and signaling of many classes of cellular targets, and the behavior of cells or cell systems. Such non-invasive and label-free cell assays can also be achieved using other label-free evanescent wave-based biosensors, such as surface plasmon resonance (SPR) or resonant mirrors. A photonic crystal biosensor is also an example of a resonant waveguide grating biosensor. These non-invasive biosensor-based cell assays measure an integrated cellular response in a label-free manner. The resultant optical signal, referred to as the dynamic mass redistribution (DMR) signal, can be induced by a stimulus and is non-specific in nature, relative to a specific cellular target or pathway. Linking a stimulus-induced optical or DMR signal to a specific target may require knowledge of, for example, pathways, cellular events, or cellular targets involved in the DMR signal (see Fang, Y., et al., (2005) Anal. Chem., 77: 5720-5725; and Fang, Y., et al., (2005) FEBS Lett., 579: 6365-6374).

### Biosensor Technologies

Biosensors comprise specific transducers for converting a molecular recognition event into a quantifiable signal. Based on the nature of transducers, they can be categorized into different types of biosensors, such as calorimetric, acoustic, electrochemical, magnetic, optical biosensors, or like sensors. Biosensors have realized widespread uses in examining molecular recognition or interactions in a label-free manner. Typically, a biological material (e.g., ligands, functional proteins, antibodies, etc.) can be contacted with the surface of a biosensor to form a biological layer. The interaction between a target analyte and the layer of biological material produces a change in a physical property of the transducer such as a change in the content of the resonantly reflected light. Such changes can be detected by the detector and used to directly quantify the binding of target molecules in a sample. Alternatively, a layer of cells can be brought into contact with the sensor surface. A stimulus is then introduced to react with the cells, producing a change in a physical property of the transducer. Such changes can be detected by the detector and used to quantify the responses of the live cells, which in turn, can be used an indicator of the function(s) of the stimulus or the target in the live-cell with which the stimulus reacts or interacts. Several types of biosensor technologies, primarily impedance-based electrical biosensors and evanescent wave-based optical biosensors, have recently been used to examine certain cellular activities under physiologic conditions.

### Evanescent Wave-Based Cell Assays

A variety of optical biosensors have been developed including, for example, surface plasmon resonance (SPR), resonant waveguide grating (RWG), and resonant mirrors. Among them, SPR and RWG are the most popular. Both technologies exploit evanescent waves to characterize molecular interactions or alterations of a biological layer at or near the sensor surface. The evanescent-wave is an electromagnetic field, created by the total internal reflection of light at a solution-surface interface, which typically extends a short distance, for example, about several hundreds of nanometers from the biosensor's surface into the solution with a characteristic depth, termed the penetration depth or the sensing volume.

SPR relies on a prism to direct a wedge of polarized light, covering a range of incident angles, into a planar glass substrate bearing an electrically conducting metallic film (e.g., gold) to excite surface plasmons. The resultant evanescent wave interacts with, and is absorbed by the free electron clouds in the gold layer, generating electron charge density waves (i.e., surface plasmons) and causing a reduction in the intensity of the reflected light. The resonance angle at which this intensity minimum occurs is a function of the refractive index of the solution close to the gold layer on the opposing face of the sensor surface. In contrast, RWG biosensor utilizes the resonant coupling of light into a waveguide by means of a diffraction grating. A polarized light having a range of incident wavelengths is used to directly illuminate the waveguide; light at specific wavelengths is coupled into and propagates along the waveguide. The resonance wavelength at which a maximum in-coupling efficiency is achieved is a function of the local refractive index at or near the biosensor surface. When target molecules in a sample bind to the immobilized receptors, the resonance wavelength shifts.

For cell-based assays, the live-cells rather than isolated receptors, are contacted with or brought to interact with the surface of a biosensor, generally via culturing. The cell adhesion can be mediated through several different types of contacts, for example, focal contacts; close contacts; and extracellular matrix (ECM) contacts. Each contact has its own characteristic separation distance from the surface. It is known that most of intracellular bio-macromolecules are well organized by the matrices of filament networks, and their location is highly regulated so that the cells can, for example, achieve specific and effective protein interactions, spatially separate protein activation and deactivation mechanisms, and determine specific cell functions and responses. Upon stimulation, there is often a significant relocation of cellular proteins, leading to a dynamic, directional, and directed mass redistribution, which is collectively referred to as dynamic mass redistribution (DMR). DMR can be detected by optical biosensors when it occurs within the sensing volume. The resultant DMR can be a unique physiological signal of live cells, and which signal can be useful, for example, monitoring receptor activation, studying the systems-cell biology of a receptor, examining the systems cell pharmacology of a drug candidate, or like applications. The biosensor-based cell assay methodologies of the disclosure can be applicable to broad ranges of cells, and cellular targets including GPCR, receptor tyrosine kinases, ion channels, kinases, and like targets.

### 1. Optical Biosensors

There are many types of label-free biosensors. These biosensors are mostly designed for molecular interaction analysis. A common feature is to use some sort of transducer to detect the molecular interactions at the surface-solution interface. Similar to other types of biosensors that utilize, for example, calorimetric, acoustic, electrochemical, or magnetic transducers, optical biosensors comprise optical transducers for converting a molecular recognition event into a quantifiable signal.

Many optical biosensor instruments are available commercially. They include, for example, Biacore's SPR-based systems such as BIACORE 3000 for kinetic determination of biomolecular interactions, Graffinity Pharmaceutical's Plasmon Imager for parallel binding detection, and Corning Inc.'s Epic® system for high throughput screening using standard SBS microtiter plate formats (primarily 384-well microplates).

In embodiments, the disclosure provides an optical biosensor system having multimodal detection capability suitable for, for example, an evanescent wave-based biosensor such as plasmon resonance, resonant mirror, photonic crystal biosensor, or resonant waveguide grating biosensor. These biosensors can exploit evanescent waves to characterize, for example, molecular or structural interactions, a chemo-mechano-electrical induced response of a live cell, or cell-cell interactions, at or near the sensor surface. The evanescent-wave is an electromagnetic field, created by the total internal reflection of light at a solution-surface interface, which typically extends a short distance (about hundreds of nanometers) into the solution with a characteristic depth, termed as penetration depth or sensing volume. Although commercial systems differ greatly in operating principle, throughput, sample delivery process, and applications, a common aspect of all optical biosensors is that they can measure changes in local refractive index at or very near the sensor surface.

Surface Plasmon Resonance (SPR). SPR relies on a prism to direct a wedge of polarized light, covering a range of incident angles, into a planar glass substrate having a gold thin film to excite surface plasmons. The resonant angle at which a minimum in intensity of reflected light occurs is a function of the refractive index of the solution close to the gold layer on the opposing face of the sensor surface.

Resonant waveguide grating (R WG) systems. A RWG biosensor uses the resonant coupling of light into a waveguide via a diffraction grating. Polarized light, covering a range of incident wavelengths, is used to illuminate the waveguide; light at specific wavelengths is coupled into and propagate along the waveguide. The resonant wavelength at which a maximum in-coupling efficiency is achieved is a function of the local refractive index at or near the sensor surface. For high throughput screening (HTS) and cell-based assays, a RWG biosensor provides a number of advantages. This type of biosensor with appropriate designs allows light at a nominally normal incident angle to illuminate the biosensor. Normal incident angle illumination is a significant design parameter for illuminating a large number of biosensors simultaneously. Simultaneous illumination is a desirable aspect for HTS which directly assays samples in the Society for Biomolecular Sciences (SBS; http://www.sbsonline.org) standard microtiter plates, such as 384-well microplates.

Interferometry systems. Interferometry based biosensors use a spectrometer to capture interference patterns in the reflected light from the biosensor interface. When biological molecules bind to the biosensor surface, its thickness can for example increase, and the binding can be monitored by analyzing, for example, changes in the interference pattern at the spectrometer.

### 2. Optical Biosensor-Based Imaging Systems

Optical biosensors generally employ a biosensor to monitor the binding of target molecules in a sample to the receptors immobilized on the surface of the biosensor. The binding signal obtained typically represents an average response due to the binding at a defined area, as determined by the size of illuminating beam (e.g., 200 microns) and the distance of the propagation length of the coupled light traveling within the biosensor (e.g., about 200 microns for RWG biosensor). There are several classes of optical biosensor systems available that are capable of imaging the binding of target molecules in a sample to immobilized receptors at high resolution. These systems include SPR imaging, ellipsometry imaging, and RWG imaging.

For example, SPR Imager® II (GWC Technologies Inc) uses prism-coupled SPR, and takes SPR measurements at a fixed angle of incidence, and collects the reflected light with a CCD camera. Changes on the surface are recorded as reflectivity changes. Thus SPR imaging collects measurements for all elements of an array simultaneously.

Ellipsometry can also be accomplished as imaging ellipsometry by using a CCD camera as a detector. This provides a real time contrast image of the sample, which provides information about film thickness and refractive index. Advanced imaging ellipsometer technology operates on the principle of classical null ellipsometry and real-time ellipsometric contrast imaging, using a single-wavelength ellipsometer setup with a laser as light source. The laser beam gets elliptically polarized after passing a linear polarizer and a quarter-wave plate. The elliptically polarized light is reflected off the sample, passes an analyzer and is imaged onto a CCD camera by a long working distance objective. Analysis of the measured data with computerized optical modeling leads to a deduction of spatially resolved film thickness and complex refractive index values.

Corning Incorporated has also disclosed a swept wavelength optical interrogation system based on RWG biosensor for imaging-based application. In this system, a fast tunable laser source is used to illuminate a sensor or an array of RWG biosensors in a microplate format. The sensor spectrum can be constructed by detecting the optical power reflected from the sensor as a function of time as the laser wavelength scans. Analysis of the measured data with computerized resonant wavelength interrogation modeling results in the construction of spatially resolved images of biosensors having immobilized receptors or a cell layer. The use of image sensors naturally leads to an imaging based interrogation scheme. Two dimensional label-free images can be obtained without any moving parts.

### 3. Evanescent-Wave (EW) Excited Fluorescence

Evanescent-wave excited-fluorescence can be used for probing bio-interfaces. This can typically be achieved using total internal reflection fluorescence (TIRF). Unlike epi-fluorescence, the evanescent-wave excited only the labeled molecules within the penetration depth of the field, which eliminated interference from a bulk signal. For TIRF, light is coupled into the interface of a substrate either through a prism or a high numerical aperture immersion objective. Here, through total internal reflection, light is guided through an entire length of the substrate.

When an evanescent-wave is generated through a highly confined waveguide mode or surface plasmon, the intense local field coupled with the long interaction length results in significant fluorescence enhancement compared to conventional TIRF, such as from about 10 to about 100 fold surface enhancement. For SPR, since evanescent-wave enhancement is highly dependent on the distance of the fluorophore to the metal surface, the detection tends to be inconsistent and it may even cause quenching. The quenching of the fluorophore by the metal surface (e.g., gold) is distance-dependent, which typically occurs within short distances, such as less than several nanometers. Such quenching does not occur when a RWG biosensor is used. Therefore, an EW generated from single-mode waveguide represents a most sensitive and quantitative measurement of a surface bound label, or like associated labels.

Evanescent wave (EW) enhanced fluorescence was proposed as early as 1985 (ref. 1). A state-of-the-art planar waveguide can be made of high index material such as Nb₂O₅, Ti₂O₅, TiO₂, SiN, and like materials, or a combination thereof. Light can be coupled into the waveguide though a prism or a surface grating. A waveguide grating coupler based EW-fluorescence technology has been commercialized by Zeptosens and Microvacuum (ref. 2). Zeptosen's devices employ an approach based on separation of the grating coupling region from the planar waveguide detection region. Microvacuum's technology is known as OWLS (optical waveguide lightmode spectroscopy) (ref. 3). The sensor is similar to a waveguide grating coupler, with the exception that optical detectors are located in the distal end of the planar waveguide. Light is coupled into the waveguide at a resonant incident angle. Similar schemes have also been reported (refs. 4, 5).

Planar waveguide is also an important technology for label free detection (refs. 2, 6). A minute change of refractive index in the waveguide surface is translated into a shift of resonant condition of the grating coupler. EW thus can be exploited for both surface confined fluorescent excitation and index of refraction sensing. Such a functionality has been reported (refs. 7, 8) using the OWLS chips and reader. Because of the detection scheme, OWLS is limited to single channel or at best one dimensional array detection.

Zeptosens and Novartis (refs. 9 to 12) use a separate region for coupling light into the waveguide film, such that the coupled light is then propagated within the waveguide film extending into another region (i.e., where there is no grating) for planar waveguide excitation. This design is optimized for EW fluorescence excitation, since the fluorescence is excited by a guided planar waveguide mode, rather than the leaky mode in the waveguide grating coupler. The guided mode can propagate longer distances than the leaky mode. Furthermore, bulk fluorescence is minimized in the planar waveguide section, while the waveguide grating coupler will leak a small amount of light and result in bulk fluorescence excitation. It should be noted, however, that the waveguide section of the Zeptosens chips can not be used for refractive index measurement. Nonetheless, these EW-excited fluorescence detection schemes or systems are designed for a biosensor substrate having a relatively small area or a small numbers of biosensors.

In embodiments of the disclosure the sensing area, i.e., the detection areas for both EW-excited fluorescence area and the EW label-independent are preferably located in the waveguide grating coupler region.

### 4. Label-Free Evanescent Wave Biosensor-Based Cell Assays

Researchers at the Corning Inc have been developing assays that utilize a label free optical biosensor, specifically a RWG biosensor, for probing cellular activity and cellular behavior in response to stimulation. Such label-free cell assays, referred to as Mass Redistribution Cell Assay Technologies (MRCAT), have been used for studying signaling G protein-coupled receptors, receptor tyrosine kinases, and many other cellular targets, and for screening compounds against these targets. The MRCAT is centered on a RWG biosensor. However, such assay can be realized using all types of EW-based biosensors including SPR or photonic crystal biosensors.

The ability of a RWG biosensor to be used for cell-based assays lies in the sensitivity of the evanescent wave, generated by the coupled light in the waveguide film, to a change in local mass density or distribution of cells cultured on the surface in response to the stimulation. For whole cell sensing using RWG biosensor, the sensor configuration can be approximately considered as a three-layer system: a substrate, a waveguide film in which a grating structure is embedded, and a cell layer. This is because a live-cell has large dimensions (typically tens of microns), and cells are cultured directly onto the surface of a RWG biosensor until typically high confluency is reached. The interaction of cells with the surface is primarily mediated through three types of contacts: focal, close, and extracellular matrix (ECM) contacts, where the cell membrane can be separated from the substrate by, for example, several nanometers to 100 nm or more. The biosensor exploits an evanescent wave to detect ligand-induced alterations of the cell layer at or near the sensor surface. A ligand-induced change in effective refractive index (i.e., the detected signal) is, to a first order, directly proportional to the change in refractive index of the bottom portion of cell layer according to equation (1):

ΔN═S(C)Δn_{c} (1)

where S(C) is the sensitivity to the cell layer, and Δn_{c} is the ligand-induced change in local refractive index of the cell layer sensed by the biosensor, which is directly proportional to the change in local concentrations of cellular targets or molecular assemblies within the sensing volume. This is attributed to a well-known physical property of cells where the refractive index of a given volume within cells is largely determined by the concentrations of bio-molecules, mainly proteins, which is also the basis for the contrast in light microscopic images of cells.

Thus, the detected signal is a sum of mass redistribution occurring at distinct distances away from the sensor surface, each with unequal contribution to the overall response. This is because of the exponentially decaying nature of the evanescent wave. Taking the weighed factor exp(−zᵢ/ΔZ_{c}) into account, the detected signal occurring perpendicular to the sensor surface is governed by equation (2):

$$Δ N = S ( N ) α d {\sum }_{i} Δ {C}_{i} [ {e}^{\frac{- {z}_{i }}{Δ {Z}_{C}}} - {e}^{\frac{- {z}_{i + 1}}{Δ {Z}_{C}}} ] \qquad ( 2 )$$

where ΔZ_{c} is the penetration depth into the cell layer, α is the specific refraction increment (about 0.0018 per 100 mL/g for proteins), zᵢ is the distance where the mass redistribution occurs, and d is an imaginary thickness of a slice within the cell layer. Here the cell layer is divided into an equally-spaced slice in the vertical direction.

Using the guidance of conventional pharmacological approaches to study receptor biology it has been demonstrated that when a ligand is specific to a receptor expressed in a cell system, the ligand-induced DMR signal is also receptor-specific, dose-dependent, and saturable (see e.g., Fang, Y., et al., in Anal. Chem., 2005, 77, 5720-5725; Biophys. J, 2006, 91. 1925-1940; FEBS Lett., 2005, 579, 6365-6374; J. Pharmacol. Tox. Methods, 2007, 55, 314-322; and BMC Cell Biol., 2007, e24 (1-12)). For a great number of G protein-coupled receptor (GPCR) ligands examined, the efficacies (measured by EC₅₀ values) were found to be almost identical to those measured using conventional methods reported in literature. The DMR signal is a novel physiologically relevant cellular response, and an integrated cellular response consisting of many cellular events downstream of the receptor activation. Because of its real-time kinetic nature, the DMR signal offers high information content for cell behavior and activity in response to stimulation, particularly in native cells.

### 5. Evanescent-Wave (EW) Excited Fluorescence for an Array of RWG Biosensors

The present disclosure provides methods that enable evanescent wave excited fluorescence for an array of optical biosensors, specifically RWG biosensors. The array of RWG biosensors is preferably in a microtiter plate (or microplate) format. One example is the Corning® Epic® biosensor microplate. Such microplate format makes EW fluorescence a more affordable tool, and high throughput enables parallel detection and screening.

In embodiments, the disclosure provides a system and a method that integrates the EW fluorescence and EW label-free detection into the same reader for the same biosensor. Having parallel label-dependent and label-independent measurement provides further specificity to label-free detection.

The Epic® system is an advanced high-throughput label-free platform. With the waveguide grating sensors integrated directly underneath each well, the sensor is designed to operate at, for example, about 830 nm wavelength and a near normal incident angle. Such long wavelength incident light makes it difficult to choose appropriate fluorescent tags for simultaneously EW-excited fluorescence and label-free detection since there are few, if any, commercially available fluorescent molecules falling into this excitation range. Near infrared (NIR) dye molecules with lower excitation wavelengths are commercially available. One example is a NIR dye made by LI-COR which can be excited at 790 nm. However, since their excitation wavelength is much lower than the resonant wavelength (e.g., about 830 nM) under a desired incident angle (i.e., near normal incident angle for large scale assays using an array of RWG biosensors, particularly for cell-based applications), it becomes apparent that significant modifications of current biosensor detection systems for such dual-detection, particularly for dyes with lower excitation wavelength are desired.

Commercially available Epic® biosensor platforms (Corning Inc.) can support long wavelengths (about 830 nm) for light coupling at a nominally normal angle. However, many commercially available membrane potential sensitive dyes are excited at visible wavelengths, such as from about 400 to about 650 nm. Thus, embodiments of the disclosure preferably use RWG biosensors that are capable of coupling and resonance of visible light at nominally normal angle incidence. Such a biosensor can be readily fabricated, for example, by appropriately adjusting the sensor configurations (i.e., pitches, grating depth, waveguide thickness, and waveguide materials).

Referring to the Figures, FIG. 1 shows a schematic of a resonant waveguide grating (RWG) biosensor for simultaneously detecting both evanescent wave (135)-excited fluorescence (150) and evanescent-wave (135) enabled optical signal (DMR signal) as a result of dynamic relocation of cellular matter (145) within the sensor's sensing volume (142) in a live-cell (140). The RWG biosensor (118) includes a substrate (120), a waveguide thin film having grating structure (125), and a contact surface (130). The biosensor can utilize an incident light consisting of a wide range of wavelengths (110) to illuminate the biosensor. As a result, the light at a specific wavelength or angle can be coupled into the waveguide, which propagates within the thin film and eventually reflects back. The reflected light (115) can be collected, recorded, and analyzed for the optical signal or DMR signal of cells in response to stimulation. The evanescent-wave excited-fluorescence can be recorded using, for example, a CCD camera, and analyzed for the redistribution of fluorescent molecules over time in cells. A dual-detection swept wavelength optical interrogation system can be used to collect both types of signals.

### 6. Sensor Design and Detection Schemes Enable Dual-Detection for an Array of RWG Biosensors

In embodiments, the disclosure provides methods that enhance the sensor, detection schemes, or both, and enable dual-detection for an array of RWG biosensors. The disclosure provides a CCD camera-based swept wavelength interrogation system for such a dual-detection system. This system uses a spectral imaging tool to acquire resonant images of the biosensor array at a sequence of different wavelengths. Each pixel of the spectral images contains a sensor spectrum, resulting in a virtual channel.

The sensor interrogation system generally includes four main components: 1) a tuneable laser for illuminating the biosensor in a swept wavelength fashion, such that each biosensor within the array can be illuminated simultaneously, although the resonant wavelengths may differ from sensor to sensor within the array (the laser can be passed through the illumination optics such that the laser beam is expanded to illuminate a part of or the entire sensor area); 2) a wavelength referencing interferometer that is used to dynamically measure the laser wavelength; 3) a digital camera that contains an area scan image sensor with digitized outputs, and can be used to record the spectral images as the tunable laser scans the wavelength; and 4) imaging optics, where a multi-element lense images the illuminated sensor area into the digital camera.

FIGS. 2A to 2C show exemplary schematics of configurations for biosensor systems that enable detection of both label-independent optical signals and label-dependent fluorescence signals of live-cells in response to stimulation. FIG. 2A shows an exemplary apparatus that includes an array of waveguide grating coupled sensors (118); laser source (211); a collimating lens (212) to shape the laser beam to cover the detection area; an excitation filter (213) with a bandwidth of ±1 nm, the incident angle of the filter can be adjusted to track the laser wavelength; an optical shutter (214) controls the exposure time to minimize the photo-bleaching; and a polarization controller (215) to align the polarization of the excitation beam to TM or TE orientation of the sensor (118). The apparatus can also include a beam splitter (216), imaging lenses (217, 218), a notch filter and fluorescence emission filter (219), and a CCD camera (220). The incident angle of the excitation beam can be adjusted through the beam splitter (216).

FIG. 2B shows an exemplary apparatus that includes an array of waveguide grating coupler sensors (118); laser source (211); a collimating lens (212) to shape the laser beam to cover the detection area; an excitation filter (213) with a bandwidth of ±1 nm, the incident angle of the filter can be adjusted to track the laser wavelength; an optical shutter (214) controls the exposure time to minimize the photo bleaching; a polarization controller (215) to align the polarization of the excitation beam to TM or TE orientation of the sensor (118). The apparatus can also include a beam splitter (216), imaging lenses (217, 218), notch filter and fluorescence emission filter (219), and a CCD camera (220). The incident angle of the excitation beam can be adjusted through the beam splitter (216). The apparatus can further include a fiber coupled tunable laser (232), a collimating lens (231), and a beam splitter (230). The tunable laser is used with the swept wavelength imaging optical interrogation system, where the detection optics (217, 218, and 219) and camera (220) are shared between label-free imaging and EW-fluorescence imaging. The two detection modes can be switched or interchanged within, for example, about 1 second. In this embodiment, the fluorescence signal can be used to interrogate the sensor. The wavelength or angle spectrum of the fluorescence intensity can be used to obtain the peak fluorescence and the refractive index simultaneously.

FIG. 2C shows another system and apparatus according to the disclosure that includes an array of waveguide grating coupled sensors (118); laser source (211); a collimating lens (212) to shape the laser beam to cover the detection area; an excitation filter (213) with a bandwidth of ±1 nm, the incident angle of the filter can be adjusted to track the laser wavelength; an optical shutter (214) controls the exposure time to minimize the photo-bleaching; and a polarization controller (215) to align the polarization of the excitation beam to TM or TE orientation of the sensor (118). The apparatus can also include a beam splitter (216), imaging lenses (217, 218), notch filter and fluorescence emission filter (219), and a CCD camera (220). The incident angle of the excitation beam can be adjusted through the beam splitter (216). The apparatus can further include a fiber coupled tunable laser (232), a collimating lens (231), a beam splitter (230), a dichroic mirror or dichromatic beam splitter (235), rear end lense (241), and a CCD/CMOS camera (242). The tunable laser can be used with the swept wavelength imaging optical interrogation system where the detection optics and camera can be separated from EW fluorescence imaging. The two detection modes can operate simultaneously and in parallel.

### 6.1 Sensor Modeling

Phase matching condition for the grating coupler can be expressed as in eq. (3):

$${n}_{eff} = m \frac{λ}{Λ} \pm sin ( θ ) \qquad ( 3 )$$

where n is the effective index of the waveguide, θ the incident angle, m the diffraction order, λ the wavelength, and Λ the grating pitch. The plus sign corresponds to the coupling into a forward propagating mode, and the negative sign to a reverse propagating mode. Epic® sensor can have a normal incident resonance wavelength of about 827 nm. The center wavelength of the tunable laser used in a swept wavelength interrogation system, as described, for example, in U.S. patent application Ser. No. 11/711,207, filed Feb. 27, 2007, entitled “Swept Wavelength Imaging Optical Interrogation System and Method for Using Same,” is 842 nm. The laser is coupled to the reverse propagating waveguide mode at an incident angle of about 3 degrees.

Based on eq. (3), the resonant wavelength of the sensor can be shifted to shorter wavelengths when coupled to the forward propagating waveguide mode with proper incident angle. Moving to shorter wavelengths with forward propagating mode, the waveguide may no longer be single mode. This can potentially reduce the grating diffraction efficiency. Using coupled wave analysis (RCWA) method (refs. 14, 15), numerical simulation for current Corning Epic® biosensor designs suggests that there may be a correlation between the incident angle and the resonant wavelength, depending on the mode used. A Corning® Epic® biosensor can include a Nb₂O₅ waveguide thin film with a thickness of about 150 nm, a grating pitch of about 500 nm, and a grating depth of about 50 nm.

For transverse magnetic (TM) modes, the resonant wavelength is shifted to 840 nm when incident angle is 3.23 degrees for the reverse propagating TM mode. The width of the resonance is related to the leakage coefficient of the waveguide grating coupler. The narrower the resonance the longer the coupling distance. This is typically about 200 μm for Epic® sensors. Conversely, when a forward propagating TM mode is used, the resonant wavelength shifts to the left when the incident angle increases. However, the grating resonant reflectivity starts to decay rapidly at about 788 nm, that is, a few nanometers of wavelength tuning can make a substantial difference in grating coupling efficiency. In addition, the width of the resonance in forward propagating mode is about one third (⅓) of that of the reverse propagating mode. This effect can be exploited, if desired, for further EW fluorescence enhancement.

For TE mode excitation, TE resonances can be maintained near 100% diffraction efficiency until the wavelength is reduced to 580 nm, where the incident angle is 53 degrees. Although TE resonance can maintain efficient coupling to a much lower wavelength, the resonance width is about 20 times wider than that of TM mode.

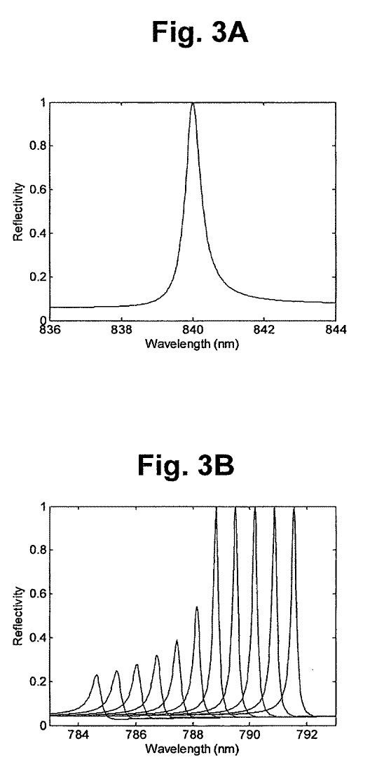

FIG. 3 shows the correlation between the resonant wavelength and the incident angle using the transverse magnetic (TM) mode. FIG. 3A shows that for the Corning® Epic® biosensor array or microplate, the resonant wavelength of a reverse propagating TM mode is 840 nm when the incident angle was 3.23°. FIG. 3B shows the inverse relation between the resonant wavelength and the incident angle when the forward propagating mode is excited. When the incident angle increases, for example, from about 3.4° with an increment of about 0.1° starting from the right, the resonant wavelength decreases.

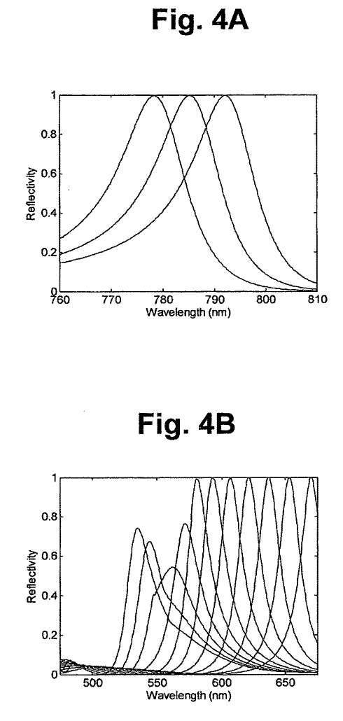

FIG. 4 shows the correlation between the resonant wavelength and the incident angle using transverse electric (TE) mode. FIG. 4A shows that for the Corning® Epic® biosensor array or microplate, the resonant wavelength of a forward propagating TE mode decreases when the incident angle increases from 16° to 17° to 18°, indicating that TE forward propagating mode using appropriate incident angles can be used to excite fluorescence of 785 nm. FIG. 4B shows that the TE mode resonances cover a much wider wavelength window with 100 percent diffraction efficiency, when the incident angle increases starting from 35° at 1° increments starting from the right.

### 6.2 Sensor Optimization for Near Infrared EW-Excited Fluorescence

The resonant enhancement of waveguide grating coupling can be understood using the Q factor given by eq. (4):

$$Q = \frac{{υ}_{0}}{Δ υ} = \frac{{λ}_{0 }}{Δλ} \qquad ( 4 )$$

where λ₀ is the resonant wavelength, Δλ the full width at half maximum of the resonance spectrum. Similar to that of an optical resonator, the larger the Q factor the longer the photon life-time and the stronger the intra-cavity electric field. Fluorescence enhancement is proportional to the Q factor and the strength of the evanescent field. As such, the enhancement for TE mode excitation is at least a factor of 20 less than that of TM mode due to its much larger leakage coefficient.

The disclosure provides methods to enhance waveguide grating biosensor designs for optimal dual detection. In embodiments, reducing the grating pitch can effectively shift the resonance wavelength to a shorter wavelength. In embodiments, keeping the same grating and reducing the waveguide thickness can also shift the resonance lower but to a smaller extent. When the waveguide thickness is reduced from 146 nm to 100 nm, the resonant wavelength of reverse propagating TM mode is moved to 785 nm. Further reduction of the waveguide thickness will result in weak guiding and a reduced evanescent field. The forward propagating mode has a narrower resonance than that of the reverse propagating mode. The sensor design can be adapted for maximum evanescent field sensitivity to provide maximum fluorescence enhancement using the forward propagating TM mode and without altering the sensor.

### 6.3 Sensor Optimization for Visible Wavelength EW-Excited Fluorescence

In embodiments, the disclosure also provides methods enabling EW-excited fluorescence of dye molecules or their conjugates with visible excitation wavelength. The method utilizes the incident angle-dependent resonant wavelength to enhance the resonant wavelength such that it enables the dual-detection system and methods. In embodiments, TE modes are used to excite shorter wavelength dyes, but with a 20-fold lower enhancement. In embodiments, TM modes with forward propagating waveguide modes are used to excite shorter wavelength dyes, but with greater incident angles. Modeling of TM mode at higher incident angles showed that the diffraction grating efficiency first decays followed by an increase when the resonant wavelength decreases from 840 nm to a visible wavelength. Although the diffraction efficiency at low resonant wavelengths is between about 40% and about 70%, the narrow resonance width suggests that the surface fluorescence enhancement is still about an order of magnitude stronger than that of TE mode excitation. In embodiments, a second order diffraction can be used to couple even shorter wavelength light. Although not limited by theory, considerable enhancement is predicted at the 400 nm region.

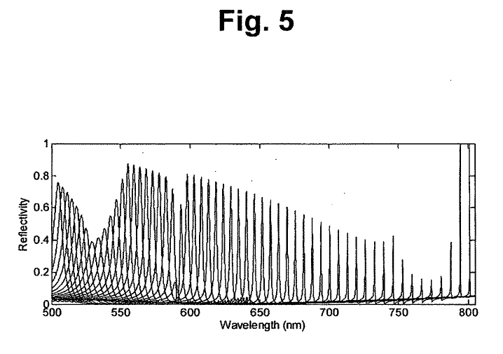

FIG. 5 shows the grating reflectivity spectra of TM modes as incident angle increases from 1 to 57 degrees, starting from the right to the left with an increment of 1 degree. Modeling of TM mode at higher incident angles shows that the diffraction grating efficiency starts to increase when the wavelength is shorter than about 750 nm. Although the diffraction efficiency is, for example, about 40% to about 70%, the narrow resonance width suggests that the surface fluorescence enhancement is still about an order of magnitude stronger than that of TE mode excitation.

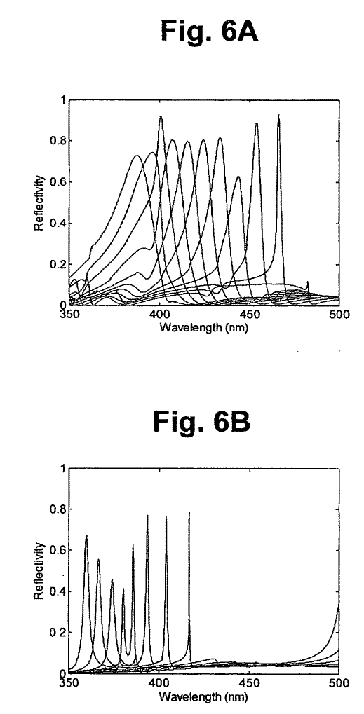

FIGS. 6A and 6B show that shorter wavelength light can be resonantly coupled into the grating through second order diffraction. FIG. 6A shows the expected resonant wavelength when a second order diffraction TE mode is used, and when the incident angle is 33° with 2° increment starting from the right. FIG. 6B shows the expected resonant wavelength when a second order diffraction TM mode is used, and when the incident angle is 41° with 2° increment starting from the right.

### 7. Dual-Detection of Cell-Signaling in Live-Cells

In embodiments, the disclosure provides methods enabling dual-detection of EW-based label-free signals (DMR signal) and EW-excited fluorescence signals in live-cells in response to stimulation. The disclosure provides methods to enhance a single resonant wavelength for both detections from a single sensor. Such parallel detection from the same biosensor having immobilized cells allows detection of cell-signaling or activity upon stimulation and provides high information content. By measuring the EW-excited fluorescence of a specific target having a label, a correlation between the target and the label-free optical signal (DMR signal) can be established. As shown among FIG. 12, using a NIR dye labeled EGF, the EW-excited fluorescence can be used for measuring the binding of the labeled EGF to the EGFR located at the basal membrane surface of the cell layer, and subsequently the internalization of the activated receptors together with the bound labeled EGF. In parallel, the EW-enabled optical response (i.e., DMR signal) of A431 cells induced by the labeled EGF can be also recorded. The results show that the labeled EGF leads to a DMR signal that is similar to that induced by unlabeled EGF (Fang, Y., et al., Biophys. J, 2006, 91, 1925-1940 (data not shown)). The DMR signal also consists of two phases: an initial increased signal (P-DMR) and a subsequent decreased signal (N-DMR).

### 8. Label-Dependent and Independent Cellular Assays for Monitoring Ion Channel Activities

In embodiments the disclosure provides a system and methods for measuring ion channel activity in living cells using an optical biosensor, particularly optical biosensors and methods based on a combination of label-dependent and independent measurements. Specifically, a biosensor having an immobilized live-cell can measure a cellular response (e.g., dynamic mass redistribution) upon ion channel activation in a label-independent manner, and can simultaneously measure the change in evanescent wave-excited fluorescence due to the redistribution of a membrane-potential sensitive label or substance, such as a membrane-potential sensitive dye molecule.

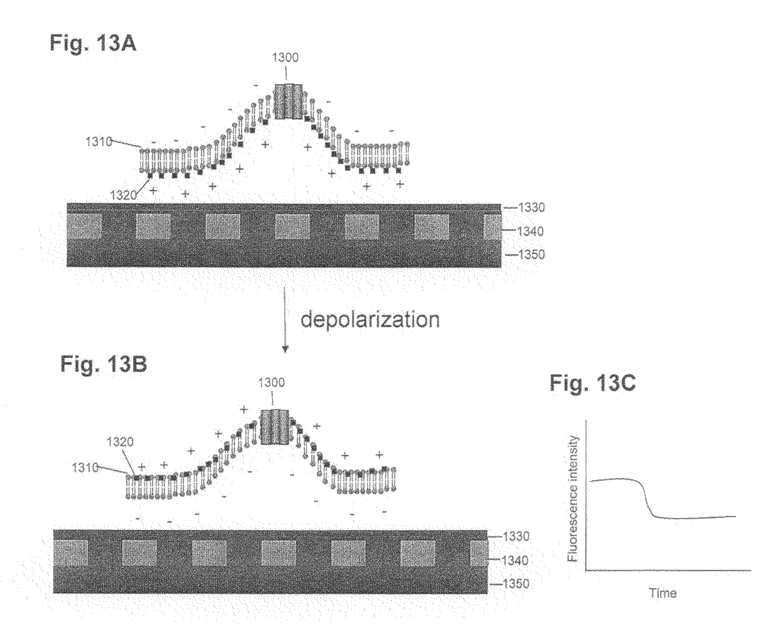

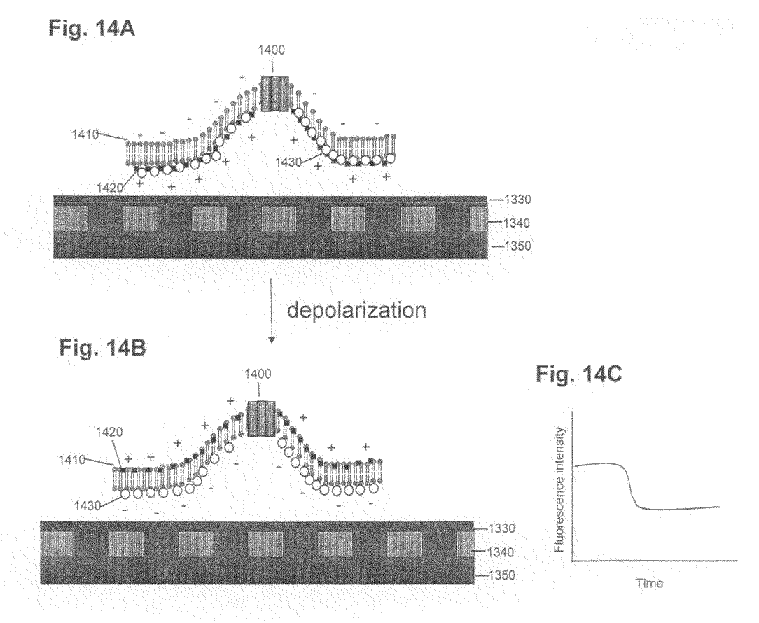

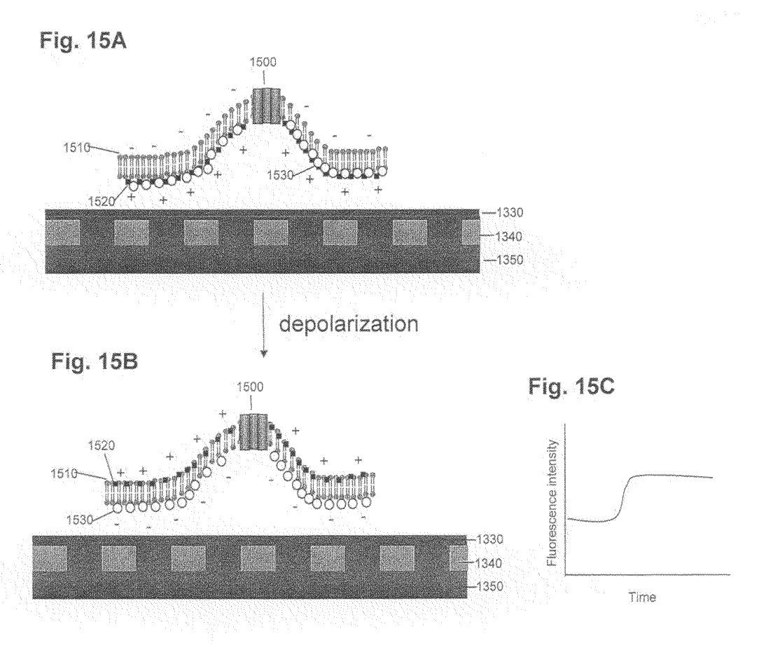

In embodiments, a live-cell can be immobilized or brought close to a biosensor surface (1330) (FIG. 13, FIG. 14, and FIG. 15) using established cell culture methods and confluencies. The biosensor can be, for example, a surface plasmon resonance (SPR) biosensor, a resonant waveguide grating (RWG) biosensor, a photonic crystal biosensor, or a resonant mirror, and like biosensors, or combinations thereof. An RWG biosensor can include, for example, a waveguide thin film having an embedded periodic grating structure (e.g., FIG. 13A, 1340), which is fused with a substrate (e.g., glass, plastic, etc.)(e.g., FIG. 13A, 1350).

FIGS. 13A to 13C shows aspects of a biosensor system and method of the disclosure that excites a membrane potential-sensitive dye in the visible region at the basal cell membrane surface that results in decreased evanescent wave-excited fluorescence due to ion-channel opening-induced cell depolarization. FIG. 13A shows a basal membrane (1310) of a live-cell having an ion channel (1300) having a visible wavelength fluorescence dye (1320) incorporated therein that is sensitive to the membrane potential. The membrane potential can be in a negative resting potential state. Because of its asymmetric distribution of charged lipid molecules in a negative resting potential state, the dye molecules are predominately located at the outside leaflet of the basal membrane bilayer. After the ion channel is opened by a physical or chemical means (e.g., a mechanical force, a voltage, a ligand, or like motives), the cell become depolarized. As shown in FIG. 13B as a result of depolarization, membrane potential sensitive dye molecules located at the outside leaflet of the lipid membrane bilayer flip to the inside leaflet, leading to a fluorescence position which is further away from the sensor surface, for example about 3 to about 5 nm. In a hypothetical example, coupled with the cell morphological changes, such flipping of membrane potential sensitive dye molecules can cause a decrease in fluorescence intensity, which can be manifested and detected by the super sensitive evanescent wave-excited fluorescence measurements. FIG. 13C shows the expected accompanying decrease in fluorescence signal intensity with respect to time for this depolarization.

In embodiments, the immobilized cells on the biosensor surface can be pre-loaded with a pair of fluorescence molecules, such as a membrane potential-sensitive dye or like substance, and a fluorescent lipid that is insensitive to membrane potential changes, either fluorescence molecule having sensitivity in at least the visible region of the excitation spectrum. FIGS. 14A to 14C show aspects of a biosensor system and method that excites a pair of fluorescence dyes, one of which is membrane potential-sensitive dye in the visible region at the basal cell membrane surface, that results in decreased evanescent wave-excited fluorescence due to ion channel opening-induced cell depolarization. The basal membrane (1410) of a cell having an ion channel (1400) can be pre-loaded with a pair of fluorescence dyes. For example, an energy donor substance such as a coumarin-linked phospholipids (CC2-DMPE) (1430) can be inserted into the outer leaflet of the cell membrane and can remain relatively stationary or localized. An energy acceptor substance (1420), such as a negatively charged oxonol dye DiSBAC2, can redistribute or change its distribution across the membrane according to the membrane potential. In a negative resting membrane potential, the acceptor dye is in close proximity to the donor dye (i.e., the outer leaflet of the basal membrane), and the energy transfer can take place. After the ion channel is opened by a physical or chemical means (e.g., a mechanical force, a voltage, a ligand, or like motive), the cell membrane becomes depolarized. The resultant change in the membrane potential with depolarization redistributes the oxonol dye (e.g., FIG. 14B; 1420) and due to the increased separation distance between the donor and acceptor, the intermolecular energy transfer is less efficient. These changes can be followed in real time by exciting the donor dye using the resonant light, and measuring the fluorescence at the emission wavelength of the acceptor dye. The depolarization-induced redistribution of the acceptor dye leads to a decrease in fluorescence, due to the lack of energy transfer from the donor dye to the acceptor. Here the sensor can be illuminated with visible wavelength light to selectively excite the donor's fluorescence, but the system measures the acceptor's fluorescence. FIG. 14C shows the expected accompanying decrease in fluorescence signal intensity with respect to time for a depolarization achieved with these initially “paired” membrane potential-sensitive dyes.

In embodiments, a cell can be pre-loaded with a membrane potential sensitive substance, such as a dye and a fluorescence quencher. FIGS. 15A to 15C show aspects of a biosensor system and method that excites a membrane potential-sensitive dye in the visible region in the presence of a fluorescence quencher. Having both dye and quencher located at the basal cell membrane surface results in increased evanescent wave-excited fluorescence due to ion channel opening-induced cell depolarization. FIG. 15A shows a basal membrane (1510) of a cell having an ion channel (1500) that is pre-loaded with a phospholipid-modified fluorescence quencher (1530), such as a nanogold phospholipid conjugate, and a membrane potential sensitive fluorescence dye (1520), such as a negatively charged oxonol dye DiSBAC2. The membrane potential sensitive dye (1520) can change its distribution across the membrane according to the membrane potential. The quencher (1530) can quench the fluorescence dye when they are in close proximity. In a negative resting membrane potential, the dye is in close proximity to the quencher (i.e., the outer leaflet of the basal membrane), and the fluorescence quenching can take place. After the ion channel is opened by a physical or chemical means (e.g., a mechanical force, a voltage, a ligand, or like motive), the cell become depolarized. FIG. 15B shows the resultant change in the membrane potential with depolarization that redistributes the potential sensitive dye. Due to the increased distance between the dye and the quencher, the quencher cannot quench the dye fluorescence. As a result, there is an increase in evanescent wave-excited fluorescence. FIG. 15C shows the expected accompanying increase in fluorescence signal intensity with respect to time for a depolarization achieved with the initially “paired” membrane potential-sensitive dye and quencher lipid.

The membrane-potential sensitive dyes can include, for example, styryl dyes, impermeant oxonol, carbocyanines, oxonols such as oxonol V and oxonol VI, and bios-oxonol dyes such as DiSBAC₂(3) or DiSBAC₄(3) (see Molecular Probes; http://www.probes.com). The fluorescence resonance energy transfer (FRET) donor can be, for example, a membrane-bound, coumarinphospholipid (CC2-DMPE), which binds only to the exterior of the cell membrane. The FRET acceptor can be, for example, a mobile, negatively charged, hydrophobic oxonol such as DiSBAC₂(3) or DiSBAC₄(3), which will bind to either side of the plasma membrane in response to changes in membrane-potential. The fluorescent quencher lipid can include, for example, nanogold particle-conjugated lipid, such as DMPE lipid. The conjugate can be made using, for example, conventional covalent coupling chemistry, such as conjugation using 1,2-dipalmitoyl phosphatidylethanolamine and mono-sulfo-NHS-Nanogolde (see http://www.nanoprobes.com). The nanogold selected in embodiments can preferably be tiny nanoparticles, having a diameter, for example, less than about 10 nanometers, or less than about 5 nanometers.

### EXAMPLES

The following examples serve to more fully describe the manner of using the above-described disclosure, and to set forth the best modes contemplated for carrying out various aspects of the disclosure. It is understood that these examples in no way limit the true scope of this disclosure, but rather are presented for illustrative purposes.

### Example 1

### Design and Characterization of a Biosensor System for Detection of EW-Excited Fluorescence

Materials—IRDye® 800CW labeled streptavidin was purchased from LI-COR Biosciences (Lincoln, Nebr.). Biotin ethylenediamine was obtained from Sigma Chemical Co. (St. Louis, Mo.). 384-well Epic® biochemical assay microplates were obtained from Corning Inc. (Corning, N.Y.). The Corning® Epic® 384-well biochemical assay microplate is an SBS standard 384-well microplate with an optical biosensor integrated into each well and is an integral component of the Epic® system for high-throughput label-free detection. Each sensor can be coated with a pre-activated surface chemistry based on, for example, polymeric maleic anhydride groups, which enables covalent attachment of protein targets via, for example, primary amine groups. Each well of the Epic® microplate also incorporates a dual sensor self-referencing area where the target proteins do not attach. This in-well reference enables the Epic® reader to report a result that represents only the effects of analyte binding.

Immobilization of IRdye® 800CW labeled streptavidin to the sensor—IRDye® 800CW labeled streptavidin of 50 μg/mL in 1× phosphate buffered saline was incubated with the 384-well biochemical assay microplate. In each well of the microplate, there is a pre-activated surface chemistry which consists of two regions: a first region being pre-reacted with ethanolamine (HOCH₂CH₂NH₂) that acts as a non-binding and negative control region; and a second reactive region which can be used to covalently interact with streptavidin through an amine-anhydride reaction.

Optical system—An optical detection system such as shown in FIG. 2A was constructed and used for dual-detection of both EW-based labeled-free signal and EW-excited fluorescence of IR dye molecules.

### Results and Discussions

IRDye® 800CM is a near-infrared (NIR) dye with a maximum excitation at 800 nm. FIG. 2A shows a system used for EW-excited fluorescence detection. The system consists of an excitation laser, an aspheric lens to collimate the laser into a parallel beam, an optical shutter synchronized with a CCD camera. The excitation laser was a laser diode with a nominal wavelength of 785 nm, matching the peak absorption of the IR dye. Maximum output power of the laser was 120 mW, although in this experiment only about 10% of the power was typically used. The laser was linearly polarized with single spatial mode. An aspheric lens was used to collimate the laser into a parallel beam, the diameter of which was matched to the field of view of the fluorescent imaging lens. In this instance the area of interest was a single grating sensor of 2×2 mm². An optical shutter was used in synchronization with the CCD camera. Alternatively, the laser power can be directly turned on and off by the driving current. Limiting the exposure time to the laser can be important for labels that are prone to photo-bleaching. The laser diode was mounted on a thermoelectric temperature block. At room temperature, the wavelength was 785 nm. When heated to 38° C., the wavelength was tuned to 790 nm. A CCD camera (Basler A102f) was chosen for fluorescence detection. The camera used a Sony ICX-285 CCD chip, which has a pixel size of 6.45 μm×6.45 μm and a full resolution frame of 1392×1040 pixels. Quantum efficiency of the image sensor at 800 nm as indicated by the manufacturer was lower than that in the visible wavelengths. With the 2× magnification imaging system, the system had a spatial resolution of about 3.2 microns/pixel.

Since 785 nm was a wavelength for which commercial off-the-shelf fluorescence filters are available, both notch filter and emission filter can maintain similar performance when the laser wavelength was tuned to 790 nm. However, the excitation filter only had a 2 nm of bandwidth. An 808 nm laser line filter was used instead. When used with an incident angle of about 16 degrees, the transmission peak of the filter was shifted to 790 nm. The system background level was as low as the 785 nm configuration.

The fluorescent system was characterized using labeled streptavidin and biotin immobilized on an Epic® plate at 785 nm wavelength. The plate had a printed blocker region. The incident angle of the excitation beam was adjusted while observing the grating with an IR viewer.

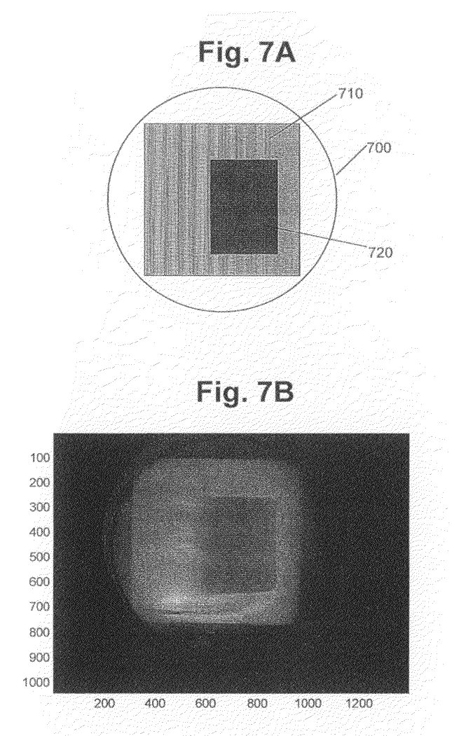

FIG. 7A shows a schematic of a RWG biosensor in a microplate array format. The biosensor was located within a well of the microplate (700), and consisted of two regions: a non-binding reference region (720) and a binding region (710). The binding region was capable of covalently coupling with an amine presenting protein or molecule such as streptavidin. FIG. 7B shows a fluorescent image of a biosensor well having dual regions using a forward propagating TM mode with a resonant wavelength of 785 nm. The image was obtained 10 min. after the incubation with the dye-labeled streptavidin and without any washing. The darker rectangular region was formed due to the ethanolamine pre-blocking induced resistance of dye-labeled streptavidin binding to this area. The brighter region was formed due to the immobilization of dye-labeled streptavidin.

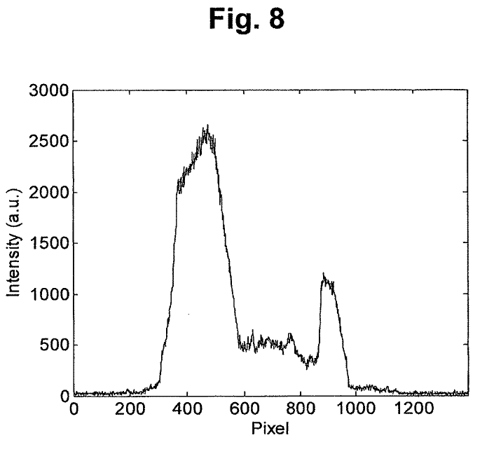

FIG. 8 shows the fluorescent intensity distribution across a row (i.e., the row at the 500^{th} pixel on the y-axis) of the same sensor in FIG. 7B after washing with a phosphate-buffered saline (PBS; 137 mM sodium chloride, 2.7 mM potassium chloride, 0.5 mM magnesium chloride (hexahydrate), 8.1 mM sodium phosphate (monobasic, monohydrate), 0.9 mM calcium chloride, and 1.47 mM potassium phosphate (monobasic, anhydrous), pH 7.2) to remove dye-labeled streptavidin in the bulk solution. Washing the well eliminates the bulk contribution, leaving only the surface bound labels. Evanescent-wave enhancement was measured by comparing the fluorescent image intensity when the excitation light was tuned in- and out-of resonant coupling angle. The results showed that the EW-excited fluorescent enhancement was about a factor of 20.

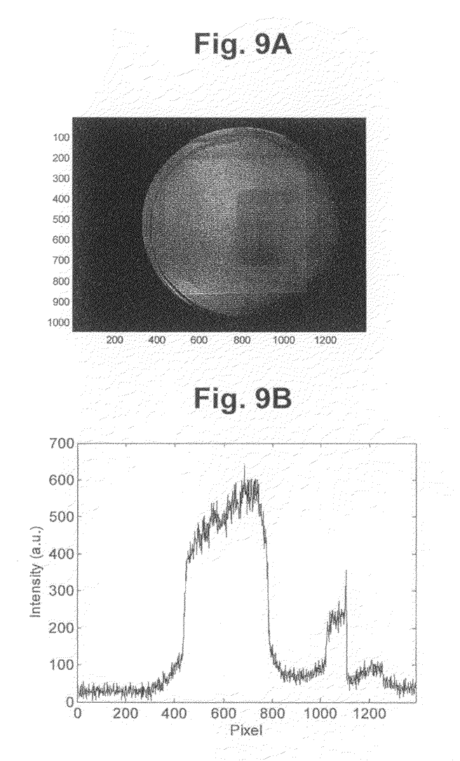

FIG. 9A shows a fluorescent image of a biosensor well having dual regions using forward propagating TE (transverse electric) mode with a resonant wavelength of 785 nm. The image was obtained 10 min. after the incubation with the dye-labeled streptavidin without any washing. The darker region was formed due to the ethanolamine preblocking-induced resistance of dye labeled streptavidin binding to this area. The brighter region was formed due to the immobilization of dye labeled streptavidin. Forward propagating TE mode was excited when the incident angle was increased to about 17° (the resonant wavelength was 785 nm). FIG. 9B shows the fluorescent intensity across the biosensor at the row position of pixel 500 in FIG. 9A. As shown here, the fluorescence from the grating surface was only marginally stronger than that from the bulk solution. After washing away the labels in the solution, the fluorescence analysis suggested that the enhancement was only about 3-fold. The low enhancement factor of TE mode was consistent with the modeling predictions.

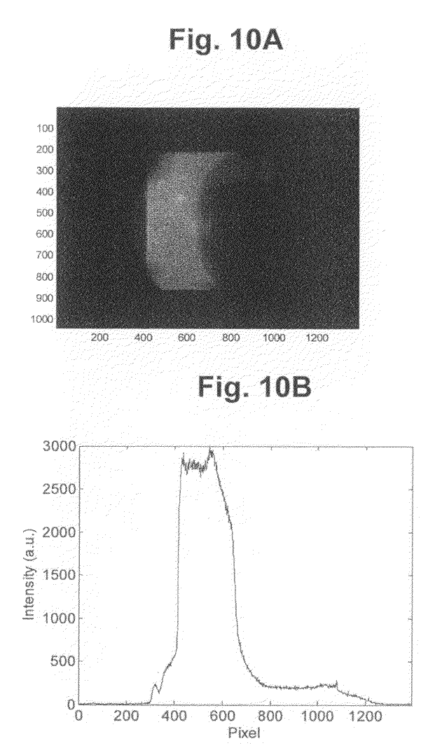

FIG. 10A shows a fluorescent image of a biosensor well having dual regions using forward propagating TM mode with a resonant wavelength of 790 nm. The imaging was obtained 10 min. after the incubation with the dye-labeled streptavidin, followed by washing. The darker region was formed due to the ethanolamine preblocking-induced resistance of dye-labeled streptavidin binding to this area, where the brighter region was formed due to the immobilization of dye-labeled streptavidin. FIG. 10B shows the fluorescent intensity distribution across the sensor at the pixel position of 500 (y-axis). Results showed that although with only 5 nm wavelength difference, the grating reflectivity became significantly stronger. As a result, the evanescent enhancement was increased to about 70, compared to the previous value of about 20 when excited at 785 nm. Fluorescence images of unwashed wells indicate that the surface signal was more than 10 times stronger than that from the bulk (data not shown). Consistent with the resonant coupled wave analysis (RCWA) modeling, the grating diffraction efficiency can be improved by a factor of about 3, reference the increased contrast between the non-binding and binding regions, when the wavelength was shifted from 785 nm to 790 nm by fine tuning the laser wavelength using a thermoelectric temperature block.

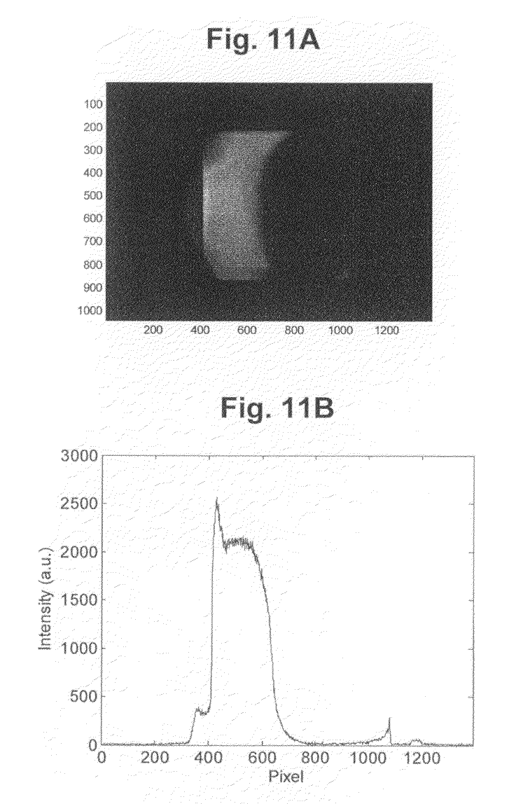

FIG. 11A shows a fluorescent image of a biosensor well having dual regions using forward propagating TM mode with a resonant wavelength of 790 nm. The imaging was obtained 10 min. after the incubation with the dye-labeled streptavidin, without any washing. FIG. 11B shows the fluorescent intensity distribution across the sensor at pixel position 500 (y-axis). The fluorescence images or intensity of unwashed wells indicate that the surface signal was more than 10 times stronger than that from the bulk.

### Example 2

Ew-excited fluorescence of IRDye® labeled epidermal growth factor (EGF) interacting with a human cancer cell line A431 Epidermal growth factor (EGF) receptor belongs to the receptor tyrosine kinase (RTK) family and is expressed in virtually all organs of mammals. EGF receptors (EGFRs) play a complex role in cell growth and differentiation, and in the progression of tumors. EGFR is also a critical downstream element of other signaling systems, and cross-talks with other receptors such as mitogenic G protein-coupled receptors (GPCRs).

EGF binds to and stimulates the intrinsic protein-tyrosine kinase activity of EGFR, initiating signal transduction cascades, principally involving the MAPK, Akt and JNK pathways. The primary event includes the binding of EGF to its cognate receptor EGFR at the cell surface membrane. Binding of EGF mediates receptor dimerization and subsequent autophosphorylation of the receptor on tyrosine residues of the cytoplasmic domain. A multitude of signaling proteins are then recruited to the activated receptors through phosphotyrosine-specific recognition motifs, including receptor internalization. During the receptor internalization, the bound EGF is also internalized.

Materials—IRDye® 800CW EGF Optical Probe (IRDye-EGF) is a near-infrared (NIR) labeled recombinant human epidermal growth factor (EGF) that was obtained from LI-COR Biosciences (Lincoln, Nebr.) (www.licor.com). The IRDye-EGF is a recombinant EGF polypeptide containing 54 amino acid residues (molecular weight=6.2 kDa) conjugated with the IRDye® fluorophore via, for example, a reactive NHS ester group that provides functionality for labeling primary and secondary amino groups. 384-well Epic® cell assay microplates were obtained from Corning Inc. (Corning, N.Y.). The surface of each Epic® cell assay microplate is tissue culture compatible and enables the attachment and normal growth of adherent cells, including native cells, recombinant or engineered cell lines, primary cells, and like cells.

Cell culturing—Human epidermoid carcinoma A431 cells (American Type Cell Culture) were grown in Dulbecco's modified Eagle's medium (DMEM) supplemented with 10% fetal bovine serum (FBS), 4.5 g/liter glucose, 2 mM glutamine, and antibiotics. About 1.5 to about 2×10⁴ cells at passage 3 to 15 suspended in 200 microliters of the DMEM medium containing 10% FBS were placed in each well of a 384-well microplate. After cell seeding, the cells were cultured at 37° C. under air/5% CO₂ until about 95% confluency was reached (about 1-2 days). The confluent cells were washed with serum-free medium and incubated in the same medium at 37° C. under air/5% CO₂ for 20 hours. On the day of assay, the cells were washed with HBSS (Hanks Balanced Salt Solution with 20 mM HEPES) buffer. The resulting A431 cells were assayed without or with pretreatment with modulators including, for example, AG1478, dynamin inhibitory peptide (DIPC), or unlabeled EGF. The EGFR was activated with the IRDye® labeled EGF and the resultant DMR signals were then recorded.

Optical system—An optical detection system is constructed and used for dual-detection of both EW-based labeled-free signal and EW-excited fluorescence of IR dye molecules as shown in FIG. 2C.

### Results and Discussion

IRDye® labeled recombinant human epidermal growth factor (IRDye-EGF), was tested for its ability to trigger receptor signaling using the MRCAT assays (data not shown). The results showed that IRDye-EGF was active and triggered cell signaling mediated through endogenous EGFRs in A431 cells, leading to a DMR signal that is similar to that induced by unlabeled EGF, but with an apparent potency of about 40 nM. Epidermal growth factor receptor (EGFR) is one of a family of receptor tyrosine kinases found on the surface of epithelial cells, to which EGF binds.

FIGS. 12A to 12D show measured fluorescence intensities of A431 cells cultured on the biosensor microplate surfaces in response to stimulation with IRDye® labeled EGF (64 nM), as a function of time. The data were generated using quiescent A431 cells under different conditions such as the cells being pre-treated with distinct reagents: FIG. 12A shows A431 cells without any pretreatment; FIG. 12B shows A431 cells pretreated with dynamin inhibitory peptide (DIPC) of 25 micromolar for 1 hr; FIG. 12C shows A431 cells pretreated with AG1478 of 10 micromolar for 1 hr; and FIG. 12D shows A431 cells pretreated with unlabeled EGF of 32 nM for 1 hr.

FIG. 12A shows the response of A431 cells without any pretreatment. Results showed that there are two major events: an initial increase in fluorescence; and thereafter by a slow decrease in fluorescence. The increase in fluorescence suggests that the dye-labeled EGF binds to the basal cell surface membrane, which is within the sensing volume such that its fluorescence is enhanced by the evanescent wave. The subsequent decrease in fluorescence is possibly due to the internalization of the bound dye-labeled EGF together with the receptor. To test this, the A431 cells were pretreated with three compounds: AG1478 which is an EGFR tyrosine kinase inhibitor; dynamin inhibitory peptide control (DIPC) which is a cell permeable dynamin inhibitor; and unlabeled EGF which can cause receptor internalization and desensitization to the subsequent stimulation with labeled EGF. AG1478 and EGF were obtained from Sigma Chemical Co. (St. Louis, Mo.) (sigmaaldrich.com), while DIPC was obtained from Tocris Chemical Co. (St. Louis, Mo.).

FIG. 12B shows that the pretreatment of A431 cells with DIPC almost completely blocked the decay phase in fluorescence intensity. This suggests that the decay phase is indeed due to the receptor internalization. Dynamin is known to play important roles in EGFR endocytosis. The blockage of dynamin activity is known to impair receptor endocytosis.

FIG. 12C shows that the pretreatment of A431 cells with AG1478 completely blocked the decay phase in fluorescence intensity, and but has complicated effects on the initial increase phase. This is consistent with EGFR receptor tyrosine kinase activity being required for EGFR signaling and internalization. The blockage of its kinase activity not only impairs the receptor signaling including internalization, but also affects the binding affinity of EGF to the receptors.

FIG. 12D shows that the pretreatment of A431 cells with unlabeled EGF (32 nM) almost completely blocked both the increase and decrease phase in fluorescence intensity. The initial increase in fluorescence intensity was partially due to bulk fluorescence, and partially due to the binding of dye-labeled EGF to the receptor. The complete inhibition of the decay phase suggests, although not limited by theory, that the EGF-treated cells become desensitized to the dye-labeled EGF.

Taken together the foregoing results suggest that the EW-excited fluorescence allows the detection of two major events associated with the dye labeled EGF interacting with the cells: the binding of fluorescently labeled EGF to the receptors located at the basal cell membrane of cultured cells, and the internalization of receptors together with the bound fluorescent EGF. Interestingly, the time that internalization takes place is consistent with our previous findings for the transition time from the P-DMR to the N-DMR event using Epics cell assays (see Fang Y., et al., Biophys. J, 2006, 91, 1925-1940), and the parallel label-free DMR signals detected with the same system (data not shown). These results confirmed that in an EGF-mediated DMR signal in fully quiescent A431 cells the P-DMR is indeed due to the recruitment of intracellular targets to activated receptors, whereas the N-DMR is due to a decrease in cell adhesion (primary), and receptor internalization (minor). The transition time may be associated with the regulatory mechanism of receptor desensitization.

The disclosure has been described with reference to various specific embodiments and techniques. However, it should be understood that many variations and modifications are possible while remaining within the spirit and scope of the disclosure.

What is claimed is:

1. A system for evanescent-wave label-free light and evanescent-wave excited-fluorescence light detection, the system comprising:
an optical sensor;
a light source to illuminate the sensor;
an optical detector to collect the evanescent-wave label-free light and the evanescent-wave excited-fluorescence light emitted from the sensor; and
a processor to analyze the collected light.

2. The system of claim 1, wherein the optical sensor comprises an array of waveguide grating coupled sensors on a microplate.

3. The system of claim 1, wherein the optical sensor has at least one live-cell immobilized on the sensor surface.

4. The system of claim 1, wherein the light source comprises a fiber coupled tunable laser system having wavelengths from about 400 to about 900 nanometers.

5. The system of claim 1, wherein the optical detector includes a self-referenced interferometer.

6. The system of claim 1, wherein the optical detector comprises a first beam splitter that adjusts the incident angle of the light source's beam and a second beam splitter that selects evanescent-wave label-free reflected light and evanescent-wave excited fluorescent label emitted light.

7. The system of claim 1, wherein the optical detector comprises a first digital camera for collecting evanescent-wave label-free reflected light and a second digital camera for collecting evanescent-wave excited-fluorescent label emitted light.

8. The system of claim 1, wherein the optical detector comprises at least one of: a collimating lens; an excitation filter optionally having a bandwidth of ±1 nm; an optical shutter; a polarization controller; an imaging lense; a notch filter; a fluorescence emission filter; or a combination thereof.

9. A method for characterizing a live-cell, the method comprising:
providing the system of claim 1 having a live-cell immobilized on the sensor's surface;
contacting the immobilized cell with a first fluorescent-labeled stimulus;
detecting the effect of the first fluorescent-labeled stimulus contact on a selected cellular target by interrogating the sensor for evanescent-wave label-free light and evanescent-wave excited-fluorescent label-emitted light; and
comparing the sensor's evanescent wave label-free light and evanescent wave excited-fluorescent label-emitted light in the presence and absence of a second stimulus.

10. The method of claim 9, wherein the fluorescent-labeled stimulus has an affinity for at least one target associated with the live-cell immobilized on the sensor's surface.

11. The method of claim 9, wherein interrogating the sensor excites the fluorescent-labeled stimulus having an association with the basal cell membrane surface of the immobilized live-cell on the surface of the sensor.

12. The method of claim 9, wherein interrogating the sensor provides evanescent-wave label-free light associated with a dynamic mass redistribution event of the immobilized live-cell.

13. The method of claim 9, wherein interrogating the sensor for evanescent-wave fluorescence and evanescent-wave label-free light is accomplished sequentially, simultaneously, or a combination thereof.

14. The method of claim 9, wherein the sensor is a resonant waveguide grating biosensor, a surface plasmon resonance, a photonic crystal biosensor, or a resonant mirror.

15. A method for characterizing a live-cell, the method comprising:
providing the system of claim 1 having a live-cell immobilized on the sensor's surface, the live-cell having a fluorescent target;
contacting the immobilized cell with a stimulus;
detecting the stimulus induced changes on the fluorescent target by interrogating the sensor for evanescent-wave fluorescence light; and
detecting the stimulus induced changes in the evanescent-wave label-free light.

16. The method of claim 15, wherein the live-cell having a fluorescent target is accomplished with a gene expression vector which expresses a fluorescent protein.

17. The method of claim 16, wherein the live-cell having a fluorescent target is accomplished with transfection, insertion of a lipid target into the cell surface membrane, or combination thereof.

18. A dual-detection system for evanescent-wave label-free light and evanescent-wave excited-fluorescence light detection, the system comprising:
an optical sensor;
a light source to illuminate the sensor;
a first optical detector to collect the evanescent-wave label-free light from the sensor;
a second detector to collect evanescent-wave excited-fluorescence light from the sensor; and
a processor to analyze the collected light.

19. The dual-detection system of claim 18, wherein the optical sensor comprises a patterned reference region, a sample region having a live-cell or a biomolecule thereon, or a combination thereof.

20. A method to enhance detection of a single resonant wavelength of an evanescent-wave label-free signal and an evanescent-wave excited-fluorescence signal from a single sensor, the method comprising:
measuring the evanescent-wave excited-fluorescence signal of a specific target having a fluorescent label, and measuring the label-free dynamic mass redistribution signal upon stimulation; and
correlating the measured fluorescence signal from the target and the label-free dynamic mass redistribution signal.

21. The method of claim 20, wherein correlating the fluorescence signal and the label-free dynamic mass redistribution signal comprises, at least one of:
comparing the kinetic profiles of both signals;
comparing the modulation profiles of both signals by alteration of signaling cascades;
comparing the impact of a gene alteration on the cellular response;
or a combination thereof.

22. A method for dual-detection of ion-channel activity in a live-cell, the method comprising:
providing a biosensor having at least one live-cell immobilized on the biosensor surface;
furnishing the immobilized cell with a membrane-potential sensitive dye;
contacting the immobilized cell having the membrane-potential sensitive dye with a stimulus; and
detecting the stimulus-induced optical label-free signal and evanescent wave excited fluorescence signal.

23. A method for dual-detection of ion-channel activity in a live-cell, the method comprising:
providing a biosensor having at least one live-cell immobilized on the biosensor surface;
furnishing the immobilized live-cell with a membrane-potential sensitive dye and a fluorescent lipid;
contacting the immobilized cell having the dye and the lipid with a stimulus; and
detecting the stimulus-induced optical label-free signal and evanescent wave excited fluorescence signal, the fluorescence signal changes in relation to a change in fluorescent resonant energy transfer between the dye and the lipid.

24. A method dual-detection of ion-channel activity in a live-cell, the method comprising:
providing a biosensor having at least one live-cell immobilized on the biosensor surface;
furnishing the immobilized cell with a membrane-potential sensitive dye and a quencher lipid;
contacting the immobilized cell having the dye and the quencher lipid with a stimulus; and
detecting the stimulus-induced optical label-free signal and evanescent wave excited fluorescence signal, the detected fluorescence signal changes in relation to a change in distance between the quencher lipid and the membrane-potential sensitive dye.
